(19) United States
(12) Patent Application Publication
(10) Pub. No.: US 2021/0315940 A1
(43) Pub. Date: Oct. 14, 2021

(54) USE OF MESENCHYMAL STROMAL CELL EXOSOMES IN ANTENATAL THERAPY

(71) Applicant: Children's Medical Center Corporation, Boston, MA (US)

(72) Inventors: Stella Kourembanas, Newton, MA (US); S. Alexander Mitsialis, Newton, MA (US); Elizabeth S. Taglauer, Brookline, MA (US)

(73) Assignee: Children's Medical Center Corporation, Boston, MA (US)

(21) Appl. No.: 17/273,684

(22) PCT Filed: Sep. 5, 2019
(86) PCT No.: PCT/US2019/049796

§ 371 Date: Mar. 4, 2021

Related U.S. Application Data
(60) Provisional application No. 62/830,023, filed on Apr. 5, 2019.
(60) Provisional application No. 62/727,222, filed on Sep. 5, 2018.

(51) Int. Cl. A61K 35/51; A61P 15/06

(52) U.S. Cl. CPC A61K 35/51; A61P 15/06

(57) ABSTRACT

Provided herein are methods of using mesenchymal stem cell (MSC) exosomes to treat placental insufficiency and/or infertility in a female subject, and/or treating fetal growth restriction in a fetus.

### RELATED APPLICATIONS

This application claims the benefit under 35 U.S.C. § 119(e) to U.S. Provisional Application No. 62/727,222, filed Sep. 5, 2018, and entitled “USE OF MESENCHYMAL STROMAL CELL EXOSOMES IN ANTENATAL THERAPY,” and U.S. Provisional Application No. 62/830,023, filed Apr. 5, 2019, and entitled “USE OF MESENCHYMAL STROMAL CELL EXOSOMES IN ANTENATAL THERAPY,” the entire contents of each of which are incorporated herein by reference.

### BACKGROUND

To date, much of the research on neonatal disease has centered on the contribution of post-natal insults. However, emerging evidence suggests that placental insufficiency (e.g., that occurs in preeclamptic pregnancies) primes the developing fetus for further injury from post-natal exposures and is associated with increased rates of disease in the neonatal period as well as in later childhood, including cardiovascular, respiratory, and metabolic disorders. Preeclamptic disease and its complications in the fetus/neonate remain highly difficult to treat.

### SUMMARY

The present disclosure is based, at least in part, on the novel finding that mesenchymal stem cell (MSC) exosomes can ameliorate harmful intrauterine environment (e.g., that caused by preeclampsia-associated placental insufficiency and inflammation) during pregnancy through immunomodulatory pathways, thereby improving pregnancy outcomes, reversing fetal growth restriction, and improving fetal health. It was also surprisingly found that, the MSC exosomes also resulted in a reversal of systemic preeclamptic symptoms in the mother.

Accordingly, some aspects of the present disclosure provide methods of treating placental insufficiency in a female subject, the methods comprising administering to the subject an effective amount of a mesenchymal stem cell (MSC) exosome.

In some embodiments, the isolated MSC exosome is isolated from MSC-conditioned media. In some embodiments, the MSC is from Warton's Jelly or bone marrow.

In some embodiments, the female subject is a human subject. In some embodiments, the female subject has preeclampsia. In some embodiments, the female subject has intrauterine inflammation. In some embodiments, the female subject has infertility.

In some embodiments, the placental insufficiency results in fetal growth restriction and/or fetal loss.

In some embodiments, the MSC exosome is administered once. In some embodiments, the MSC exosome is administered repeatedly. In some embodiments, the MSC exosome is administered via intravenous injection. In some embodiments, the MSC exosome is administered via intrauterine injection. In some embodiments, the MSC exosome is administered antepartum. In some embodiments, the MSC exosome is administered intrapartum.

In some embodiments, the MSC exosome reduces intrauterine inflammation. In some embodiments, the MSC exosome reverses placental insufficiency. In some embodiments, the MSC exosome reduces the likelihood of fetal growth restriction and/or fetal loss.

Further provided herein are the use of a mesenchymal stem cell (MSC) exosome to treat placental insufficiency in a female subject.

Other aspects of the present disclosure provide methods of treating fetal growth restriction, the methods comprising administering to a fetus in a pregnant female subject an effective amount of a mesenchymal stem cell (MSC) exosome.

In some embodiments, the isolated MSC exosome is isolated from MSC-conditioned media. In some embodiments, the MSC is from Warton's Jelly or bone marrow.

In some embodiments, the fetus is a human fetus. In some embodiments, the fetal growth restriction is caused by placental insufficiency of the pregnant female subject.

In some embodiments, the MSC exosome is administered via intravenous injection to the pregnant female subject. In some embodiments, the MSC exosome is administered to the amniotic fluid of the pregnant female subject. In some embodiments, the MSC exosome is administered via injection into the umbilical vein of the umbilical cord. In some embodiments, the MSC exosome is administered once. In some embodiments, the MSC exosome is administered repeatedly. In some embodiments, the MSC exosome is administered antenatal. In some embodiments, the MSC exosome is administered intrapartum. In some embodiments, the MSC exosome is administered perinatal.

In some embodiments, the MSC exosome reduces the likelihood of fetal loss. In some embodiments, the MSC exosome ameliorates pre-eclampsia-related alterations in fetal lung development.

Further provided herein are the use of a mesenchymal stem cell (MSC) exosome to treat fetal growth restriction of a fetus in a pregnant female subject.

Other aspects of the present disclosure provide methods of treating infertility, the methods comprising administering to a female subject in need thereof an effective amount of a mesenchymal stem cell (MSC) exosome.

In some embodiments, the isolated MSC exosome is isolated from MSC-conditioned media. In some embodiments, the MSC is from Warton's Jelly or bone marrow.

In some embodiments, the subject is a human subject. In some embodiments, the female subject has history of pelvic inflammatory disease, advanced maternal age, obesity, metabolic or cardiovascular disease, history of endometriosis or fibroids, chronic maternal hypertension, polycystic ovary syndrome, and/or history of sexually transmitted infections with secondary scarring. In some embodiments, the subject has intrauterine inflammation. In some embodiments, the subject has placental insufficiency.

In some embodiments, the MSC exosome is administered once. In some embodiments, the MSC exosome is administered repeatedly. In some embodiments, the MSC exosome is administered via intravenous injection. In some embodiments, the MSC exosome is administered via intrauterine injection.

Further provided herein are the use of a mesenchymal stem cell (MSC) exosome to treat infertility in a female subject.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A to 1C. Preeclampsia-associated fetal loss and intrauterine growth restriction are prevented by antenatal MEX administration. Mid-Pregnancy (E12) evaluation of fetal loss and fetal length in homozygous and hemizygous matings of HO-1−/− female mice. Labels: wildtype (WT), HO-1 −/− (KO), or HO-1 −/− treated with MEX (KO+MEX). FIG. 1A: Gravid uteri with arrows denoting either healthy implantation sites (IS) or sites of fetal loss, resorptions sites (RS); images of E12 fetuses, depicting crown rump length measurements. FIG. 1B: Graphical analysis of percentage of fetal loss from n=3-6 pregnant dams/group. FIG. 1C: Graphical analysis of percentage of fetal loss from n=3-6 pregnant dams/group.

FIGS. 2A to 2C. MEX therapy alters macrophage phenotype at the HO-1^{-/-} maternal-fetal interface. Flow cytometric analysis of macrophages isolated from gd12 implantation site (IS) tissues containing decidua, placental and fetal membranes (without the fetus) from experimental groups. FIG. 2A: Representative histograms of CD11c and CD40 staining in cell population from parent gate of CD45⁺, CD11b⁺, F4/80⁺ IS macrophages (parent gating not shown). FIG. 2B: Mean fluorescence intensity (MFI) of CD11c in IS macrophage population. FIG. 2C: Percentage of CD11c^{hi} CD40^{hi} cells in IS macrophage population. (* p<0.05, **p<0.01, ***p<0.001)

FIG. 3. Preeclamptic renal pathology in HO-1 −/− mothers is prevented by antenatal MEX therapy. Representative images of glomerular histology from WT, KO or MEX treated KO (KO+MEX) pregnant females at gd 12. Arrows denote areas of protein deposition disrupting glomerular architecture

FIGS. 4A to 4D. Antenatal MEx therapy attenuates placental and renal preeclamptic stigmata. Mid-Pregnancy (E12) evaluation of homozygous matings. Females: wildtype (WT), HO-1 −/− (KO), or HO-1 −/− treated with MEx (KO+Mex). FIG. 4A: H&E images of uterine spiral artery morphology representative of n=6-8 placentas per group. FIG. 4B: Quantification of blood vessel wall:lumen ratio, measurements averaged from 3-4 10× visual fields/placenta. FIG. 4C: Representative H&E images of renal glomerular histology from n=5 kidneys/experimental group. Arrows denote areas of proteinaceous material disrupting glomerular architecture. FIG. 4D: ELISA analysis of urine albumin, samples collected from n=3 pregnant dams/group.

FIGS. 5A to 5B. Postnatal Effects of Antenatal MEx Treatment. FIG. 5A: Experimental model to evaluate postnatal effects of antenatal MEx treatment. FIG. 5B: Evaluation of neonatal weight. n=12-14 mice (2-3 litters)/group.

FIGS. 6A to 6B. Neonatal Lung Morphology Following Antenatal MEx Treatment. FIG. 6A: Representative H&E images from neonatal lung histology. FIG. 6B: Quantification of lung morphology using mean linear intercept (MLI) analysis. n=6-8 lungs (2-3 litters)/group.

FIG. 7. Molecular Changes in Fetal Lung Following Antenatal MEx treatment. Quantitative (qPCR) analysis of lung development genes Nkx 2.1 and eNOS. n=5 fetal (sampled from 2-3 litters)/group. Both genes of interest were normalized to housekeeping gene Nup133 and analyzed by the 2^{-ΔΔCt} method.

FIGS. 8A to 8B. MSC-derived extracellular vesicles traffic to a specific subset of cells within the preimplantation uterus. FIG. 8A: Study design of biodistribution analysis of labeled extracellular vesicles (EV) in plug positive WT female at E1. FIG. 8B: Fluorescent images of DAPI labeled cytospins of digested uterine or kidney cell suspensions at 60× and 100× magnification. White arrows denote cells with uptake of labeled EV within uterine cell suspensions at 60× magnification. Control injection denotes tail vein injection of second wash supernatant collected during EV labeling protocol to assess for residual presence of free dye. Images representative of tissues harvested from two different females, utilizing two different preps of labeled EV or control wash supernatant.

FIGS. 9A to 9E. Mass cytometric (CyTOF) analysis highlights intrauterine myeloid and natural killer cell populations altered by antenatal MEx therapy. Immune cells isolated from E12 homozygous uterine/placental tissues in homozygous pregnancies analyzed with a 27 marker panel. Labels: wildtype (WT), HO-1 −/− (KO), or HO-1 −/− treated with MEx (KO+Mex). FIG. 9A: Hierarchical consensus cluster analysis identifying 49 distinct cell populations based on surface marker commonality. Circles indicate clusters with significant abundance changes between all experimental groups. FIG. 9B: Graphical representation of cluster abundance values. FIG. 9C: Manual gating analysis of F4/80+ population correlating with Cluster 35. FIG. 9D: Manual gating analysis of CD11c+ population correlating with Cluster 37. FIG. 9E: Quantification of uterine NK (uNK) cells based on manual gating. MSI: mean signal intensity. n=6 combined utero/placental implantation sites from 4 pregnant dams/group.

FIG. 10. Multi-cellular cytokine profiles altered in preeclampsia are normalized by antenatal MEx therapy. Labels: wildtype (WT), HO-1 −/− (KO), or HO-1 −/− treated with MEx (KO+Mex). Combined cytokine analysis from relative mean signal intensity from CyTOF intracellular cytokine analysis of utero-placental tissues at E12.

FIGS. 11A to 11E. Preeclampsia-associated alterations in lung development are ameliorated by antenatal Mex treatment. FIG. 11A: Analysis of developmental gene expression in E17 fetal lungs from hemizygous pups with differing maternal environments as shown. Schematic of lung development highlighting main gene transcripts altered by the HO-1 −/− preeclamptic maternal environment and qPCR analysis of hemizygous E17 fetal lungs from maternal phenotypes: wild type (WT), HO-1 −/− (KO), or HO-1 −/− treated with MEx (KO+MEx). Fold change relative to WT was calculated using 2−ΔΔCT method. n=5 fetal lung samples from 2 litters/group. FIG. 11B: Evaluation of PN14 neonatal lung histology also from hemizygous pups with differing maternal environments as shown. H&E images (40× magnification) representative of n=6-8 pups, 2-3 litters/group. FIG. 11C: Quantification of average pup weight/litter, 6-8 pups/litter, 4 litters/group. FIG. 11D: Mean linear intercept analysis quantifying lung alveolarization. FIG. 11E: Pup weight at PN14.

FIGS. 12A to 12D. Amniotic fluid confers the therapeutic effect of antenatal MEx to improve fetal lung development in preeclamptic pregnancies. FIG. 12A: Experimental design for amniotic fluid:lung explant cultures and experimental analyses. Data evaluated from total of 2 separate experiments utilizing amniotic fluid from 2 different pregnancies per experimental condition. Lung explants harvested from 2-3 wild type pregnant dams per experiment, plated into 4-5 explants per condition from each experiment. FIG. 12B: Representative images of lung explants from different experimental conditions at 4× and 10× magnification. FIG. 12C: Quantification of average new branches/mm2 at end of a 72-hour culture period. FIG. 12D: qPCR analysis of RNA harvested from pooled explants from two separate experiments, run in triplicate. Fold changes relative to WT pregnancy values were calculated using 2−ΔΔCT method.

FIGS. 13A to 13F. Mesenchymal Stromal Cell (MSC) and MEx characterization. FIG. 13A: Representative 4× images of MSC under control media conditions at P3 or following exposure to differentiation conditions for chondrogenesis, adipogenesis and osteogenesis. FIG. 13B: Flow cytometric analysis of MSC purity at P2, assessing for positive and negative human MSC markers. FIG. 13C: Schematic of MEx isolation from MSC conditioned media. FIG. 13D: Western blot analysis of iodixanol fractions 1-12, highlighting exosome-specific expression of ALIX, CD63, CD81, Syntenin and negative expression of GM130 in MEx enriched fraction 9. FIG. 13E: Purified MEx from fra were additionally evaluated using nanocyte analysis, to assess particle size distribution and concentration. FIG. 13F: Electron microscopy visualization vesicle morphology and size in each prep.

FIG. 14. Representative surface heat maps from wild type pregnancy generated by FlowSOM hierarchical cluster analysis for 20 surface markers used to evaluate the CD45+ cell populations of the utero-placental interface at E12.

FIGS. 15A to 15B. FIG. 15A: Manual gating strategy for CyTOF analysis of E12 utero-placental tissues. FIG. 15B: Relative abundance of total CD45+ cells and major immune cell types based on manual gating.

### DETAILED DESCRIPTION OF CERTAIN EMBODIMENTS

The present disclosure is based, at least in part, on the novel finding that mesenchymal stem cell (MSC) exosomes can ameliorate harmful intrauterine environment (e.g., that caused by preeclampsia-associated placental insufficiency and inflammation) during pregnancy through immunomodulatory pathways, thereby improving pregnancy outcomes, reversing fetal growth restriction, and improving fetal health. It was also surprisingly found that, the MSC exosomes also resulted in a reversal of systemic preeclamptic symptoms in the mother. Provided herein are the use of MSC exosomes in treating placental insufficiency (e.g., without limitation, placental insufficiency associated with preeclampsia) and/or infertility in female subjects, and the use of MSC exosomes in treating fetal growth restriction and/or in reducing the likelihood of fetal loss.

Some aspects of the present disclosure provide methods of treating placental insufficiency in a female subject, the method comprising administering to the subject an effective amount of a mesenchymal stem cell (MSC) exosome.

“Placental insufficiency” (also termed “uteroplacental vascular insufficiency”) is a complication of pregnancy when the placenta is unable to deliver an adequate supply of nutrients and oxygen to the fetus, and, thus, cannot fully support the developing fetus. Placental insufficiency occurs when the placenta either does not develop properly or because it has been damaged. Key reasons that may lead to placental insufficiency include, without limitation: maternal vascular disease, diabetes, anemia, chronic hypertension, blood clotting disorders, maternal smoking; and previous uterine surgery with scarring leading to abnormal placentation such as placenta previa.

Placental insufficiency includes a reduction in the maternal blood supply (reduced uterine artery blood flow) and/or the failure of the maternal blood supply to increase or adapt appropriately by mid-pregnancy. Placental insufficiency can result in pregnancy complications, including fetal growth restriction, pre-eclampsia and others. Factors considered during management of complicated pregnancies are maternal medical and obstetrical history, weight, ethnicity, and blood pressure.

In some embodiments, the female subject that has placental insufficiency also has preeclampsia. “Preeclampsia” is a pregnancy complication characterized by high blood pressure and signs of damage to another organ system, most often the liver and kidneys. Preeclampsia usually begins after 20 weeks of pregnancy in women whose blood pressure had been normal. Left untreated, preeclampsia can lead to serious, even fatal complications for both the pregnant female and the fetus.

Preeclampsia sometimes develops without any symptoms. High blood pressure may develop slowly, or it may have a sudden onset. Other signs and symptoms of preeclampsia may include, without limitation: excess protein in the urine (proteinuria) or additional signs of kidney problems, severe headaches, changes in vision, including temporary loss of vision, blurred vision or light sensitivity, upper abdominal pain, usually under the ribs on the right side, nausea or vomiting, decreased urine output, decreased levels of platelets in the blood (thrombocytopenia), impaired liver function, shortness of breath caused by fluid in the lungs, sudden weight gain and swelling (edema, e.g., particularly in face and hands).

In some embodiments, the female subject having placental insufficiency has intrauterine inflammation. “Intrauterine inflammation” refers to inflammation of the chorion, amnion, and placenta. Intrauterine inflammation can be caused by bacterial infection, also referred to as chorioamnionitis. Intrauterine inflammation is one of the most common antecedents of premature birth. The incidence of intrauterine inflammation is inversely related to gestational age, such that it is implicated in the majority of extremely preterm births and 16% of preterm births at 34 weeks (e.g., as described in Lahra et al., Archives of Disease in Childhood, vol. 94, no. 1, pp. F13-F16, 2009; and Lahra et al., American Journal of Obstetrics and Gynecology, vol. 190, no. 1, pp. 147-151, 2004, incorporated herein by reference).

Placental insufficiency, preeclampsia, and intrauterine inflammation are often associated with each other. In some embodiments, intrauterine inflammation leads to placental insufficiency and preeclampsia. In some embodiments, the conditions (placental insufficiency, preeclampsia, and intrauterine inflammation) are associated with each other without a causal relationship. In some embodiments, vascular/abnormal placentation associated with hypoxia as well as the chronic inflammation can lead to preeclampsia.

Placental insufficiency, preeclampsia, and/or intrauterine inflammation, alone or together impact the health of the pregnant female and the fetus. For example, in some embodiments, placental insufficiency, preeclampsia, and/or intrauterine inflammation, alone or in combination, leads to maternal long term cardiovascular and metabolic morbidities that are associated with infertility in the female subject. “Infertility,” as used herein, refers to the inability of a female subject to become pregnant or carry a pregnancy to full term.

In some embodiments, placental insufficiency, preeclampsia, and/or intrauterine inflammation, alone or together, lead to complications in the fetus, e.g., fetal growth restriction and/or fetal loss. “Fetal growth restriction (also referred to as “intrauterine Growth Restriction”)” refers to a condition when a fetal weight is below the 10th percentile for gestational age (e.g., as determined through an ultrasound). In some embodiments, fetal growth restriction is characterized by all internal organs being reduced in size. In some embodiments, fetal growth restriction is characterized by the head and brain being normal in size, but the abdomen is smaller. “Fetal loss” refers to the death of a fetus at any time during pregnancy. For the purpose of the present disclosure, fetal loss is also a reason for infertility in the female subject.

The present disclosure demonstrates that MSC exosomes are effective in alleviating or reversing the various conditions described herein in the female subject and in the fetus. In some embodiments, the MSC exosome reduces intrauterine inflammation (e.g., by at least 20%), compared to in the absence of the MSC exosomes. For example, the MSC exosome may reduce intrauterine inflammation by at least 20%, at least 30%, at least 40%, at least 50%, at least 60%, at least 70%, at least 80%, at least 90%, at least 95%, at least 99% or more, compared to in the absence of the MSC exosomes. In some embodiments, the MSC exosome reduces intrauterine inflammation by 20%, 30%, 40%, 50%, 60%, 70%, 80%, 90%, 95%, 99% or more, compared to in the absence of the MSC exosomes. One skilled in the art is familiar with markers that indicate intrauterine inflammation. For example, as demonstrated herein, CD11c and CD40 are indicators of intrauterine proinflammatory macrophage phenotypes.

In some embodiments, the MSC exosome reverses placental insufficiency. “Reverses placental insufficiency” means alleviating or eliminating the symptoms of placental insufficiency in the female subject or alleviating or eliminating the consequence of placental insufficiency in the fetus. For example, in some embodiments, the MSC exosome reduces the likelihood of fetal growth restriction. For example, the MSC exosome may reduce the likelihood of fetal growth restriction by at least 20%, at least 30%, at least 40%, at least 50%, at least 60%, at least 70%, at least 80%, at least 90%, at least 95%, at least 99% or more, compared to in the absence of the MSC exosomes. In some embodiments, the MSC exosome reduces the likelihood of fetal growth restriction by 20%, 30%, 40%, 50%, 60%, 70%, 80%, 90%, 95%, 99% or more, compared to in the absence of the MSC exosomes. In some embodiments, the MSC exosome reverses fetal growth restriction. “Reverse fetal growth restriction” means the fetus that is suffering from fetal growth restriction develops normal sized organs, head, and/or brain, after receiving treatment with MSC exosomes.

In some embodiments, treating the fetus using the MSC exosomes reduces the impact of fetal growth restriction on the development and health of the fetus at a later stage (e.g., when the fetus is born, in adolescence, and/or in adulthood). For example, the fetus treated with the MSC exosomes may be less likely (e.g., at least 20%, at least 30%, at least 40%, at least 50%, at least 60%, at least 70%, at least 80%, at least 90%, at least 95%, or at least 99% less) to develop a disease associated with fetal growth restriction (e.g., underdeveloped organs, premature birth, etc.).

In some embodiments, the MSC exosome reduces the likelihood of fetal loss (e.g., by at least 20%), compared to in the absence of the MSC exosomes. For example, the MSC exosome may reduce the likelihood of fetal loss by at least 20%, at least 30%, at least 40%, at least 50%, at least 60%, at least 70%, at least 80%, at least 90%, at least 95%, at least 99% or more, compared to in the absence of the MSC exosomes. In some embodiments, the MSC exosome reduces the likelihood of fetal loss by 20%, 30%, 40%, 50%, 60%, 70%, 80%, 90%, 95%, 99% or more, compared to in the absence of the MSC exosomes.

In some embodiments, the MSC exosome ameliorates pre-eclampsia-related alterations in fetal lung development. Maternal preeclampsia is associated with worse neonatal lung disease outcomes. It was demonstrated herein that, the MSC-exosomes are effective in restoring neonatal lung morphology and development for neonates that suffered fetal growth restriction due to maternal preeclampsia.

Other aspects of the present disclosure provide methods of treating infertility. The method comprising administering to a female subject in need thereof an effective amount of a mesenchymal stem cell (MSC) exosome.

In some embodiments, the female subject has been diagnosed of infertility. In some embodiments, the female subject is at risk of infertility. A female subject that is at risk of infertility may have one or more characteristics including, without limitation: history of pelvic inflammatory disease, advanced maternal age (e.g., >40 years old), obesity, metabolic or cardiovascular disease, history of endometriosis or fibroids, chronic maternal hypertension, polycystic ovary syndrome, and history of sexually transmitted infections with secondary scarring. In some embodiments, the female subject has intrauterine inflammation and/or placental insufficiency.

In some embodiments, the MSC exosomes increases the chance of the female subject in conceiving, thus treating infertility. For example, the MSC exosome may increase the chance of the female subject in conceiving by at least 20%, at least 30%, at least 40%, at least 50%, at least 60%, at least 70%, at least 80%, at least 90%, at least 100%, at least 2-fold, at least 5-fold, at least 10-fold, or more, compared to in the absence of the MSC exosomes. In some embodiments, the MSC exosome increases the chance of the female subject in conceiving 20%, 30%, 40%, 50%, 60%, 70%, 80%, 90%, 100%, 2-fold, 5-fold, 10-fold or more, compared to in the absence of the MSC exosomes.

In some embodiments, the MSC exosome reduces the likelihood of fetal loss, thus treating infertility. For example, the MSC exosome may reduce the likelihood of fetal loss by at least 20%, at least 30%, at least 40%, at least 50%, at least 60%, at least 70%, at least 80%, at least 90%, at least 95%, at least 99% or more, compared to in the absence of the MSC exosomes. In some embodiments, the MSC exosome reduces the likelihood of fetal loss by 20%, 30%, 40%, 50%, 60%, 70%, 80%, 90%, 95%, 99% or more, compared to in the absence of the MSC exosomes.

The MSC exosomes are effective in reducing the likelihood of fetal loss. Thus, the present disclosure also contemplates methods of treating fetal growth restriction, the method comprising administering to a fetus in a pregnant female subject an effective amount of a mesenchymal stem cell (MSC) exosome. In some embodiments, the fetal growth restriction is caused by placental insufficiency of the pregnant female subject.

An “exosome” is a membrane (e.g., lipid bilayer) vesicle that is released from a cell (e.g., any eukaryotic cell). Exosomes are present in eukaryotic fluids, including blood, urine, and cultured medium of cell cultures. The exosomes of the present disclosure are released from mesenchymal stem cells (MSCs) and are interchangeably termed “mesenchymal stem cell exosomes” or “MSC exosomes.”

A “mesenchymal stem cell (MSC)” is a progenitor cell having the capacity to differentiate into neuronal cells, adipocytes, chondrocytes, osteoblasts, myocytes, cardiac tissue, and other endothelial or epithelial cells. (See for example Wang, Stem Cells 2004; 22(7); 1330-7; McElreavey; 1991 Biochem Soc Trans (1); 29s; Takechi, Placenta 1993 March/April; 14 (2); 235-45; Takechi, 1993; Kobayashi; Early Human Development; 1998; Jul. 10; 51 (3); 223-33; Yen; Stem Cells; 2005; 23 (1) 3-9.) These cells may be defined phenotypically by gene or protein expression. These cells have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L1. These cells have also been characterized as not expressing (and thus being negative for) CD3, CD5, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1, and ABO. Thus, MSCs may be characterized phenotypically and/or functionally according to their differentiation potential.

MSCs may be harvested from a number of sources including but not limited to bone marrow, blood, adipose tissue, periosteum, dermis, umbilical cord blood and/or matrix (e.g., Wharton's Jelly), and placenta. Methods for harvesting MSCs are described in the art, e.g., in U.S. Pat. No. 5,486,359, incorporated herein by reference.

MSCs can be isolated from multiple sources, e.g., bone marrow mononuclear cells, umbilical cord blood, adipose tissue, placental tissue, based on their adherence to tissue culture plastic. For example, MSCs can be isolated from commercially available bone marrow aspirates. Enrichment of MSCs within a population of cells can be achieved using methods known in the art including but not limited to fluorescence-activated cell sorting (FACS).

Commercially available media may be used for the growth, culture and maintenance of MSCs. Such media include but are not limited to Dulbecco's modified Eagle's medium (DMEM). Components in such media that are useful for the growth, culture and maintenance of MSCs, fibroblasts, and macrophages include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. MSCs can be maintained using methods known in the art, e.g., as described in Pittenger et al., Science, 284:143-147 (1999), incorporated herein by reference.

In some embodiments, the MSC exosomes used to treat the conditions/diseases described herein are isolated exosomes. As used herein, an “isolated exosome” is an exosome that is physically separated from its natural environment. An isolated exosome may be physically separated, in whole or in part, from tissue or cells with which it naturally exists, including MSCs, fibroblasts, and macrophages. In some embodiments, the isolated exosomes are MSC exosomes, In some embodiments, the MSC exosomes are isolated from the culturing media of MSCs from human bone marrow, or umbilical cord Wharton's Jelly. Such culturing media is termed “MSC-conditioned media” herein. In some embodiments, isolated exosomes may be free of cells such as MSCs, or it may be free or substantially free of conditioned media, or it may be free of any biological contaminants such as proteins. Typically, the isolated exosomes are provided at a higher concentration than exosomes present in unmanipulated conditioned media.

In some embodiments, the isolated MSC exosome is substantially free of contaminants (e.g., protein contaminants). The isolated MSC exosome is “substantially free of contaminants” when the preparation of the isolated MSC exosome contains fewer than 20%, 15%, 10%, 5%, 2%, 1%, or less than 1%, of any other substances (e.g., proteins). In some embodiments, the isolated MSC is “substantially free of contaminants” when the preparation of the isolated MSC exosome is at least 80%, at least 85%, at least 90%, at least 95%, at least 98%, at least 99%, at least 99.9% pure, with respect to contaminants (e.g., proteins).

“Protein contaminants” refer to proteins that are not associated with the isolated exosome and do not contribute to the biological activity of the exosome. The protein contaminants are also referred to herein as “non-exosomal protein contaminants.”

The MSC exosome described herein has a diameter of about 30-150 nm. For example, the MSC exosome may have a diameter of 30-150, 30-140, 30-130, 30-120, 30-110, 30-100, 30-90, 30-80, 30-70, 30-60, 30-50, 30-40, 40-150, 40-140, 40-130, 40-120, 40-110, 40-100, 40-90, 40-80, 40-70, 40-60, 40-50, 50-150 nm, 50-140 nm, 50-130 nm, 50-120 nm, 50-110 nm, 50-100 nm, 50-90 nm, 50-80 nm, 50-70 nm, 50-60 nm, 60-150 nm, 60-140 nm, 60-130 nm, 60-120 nm, 60-110 nm, 60-100 nm, 60-90 nm, 60-80 nm, 60-70 nm, 70-150 nm, 70-140 nm, 70-130 nm, 70-120 nm, 70-110 nm, 70-100 nm, 70-90 nm, 70-80 nm, 80-150 nm, 80-140 nm, 80-130 nm, 80-120 nm, 80-110 nm, 80-100 nm, 80-90 nm, 90-150 nm, 90-140 nm, 90-130 nm, 90-120 nm, 90-110 nm, 90-100 nm, 100-150 nm, 100-140 nm, 100-130 nm, 100-120 nm, 100-110 nm, 110-150 nm, 110-140 nm, 110-130 nm, 110-120 nm, 120-150 nm, 120-140 nm, 120-130 nm, 130-150 nm, 130-140 nm, or 140-150 nm. In some embodiments, the MSC exosome may have a diameter of about 50 nm, 60 nm, 70 nm, 80 nm, 90 nm, 100 nm, 110 nm, 120 nm, 130 nm, 140 nm, or 150 nm. In some embodiments, the MSC exosomes exhibit a biconcave morphology.

In some embodiments, the MSC exosomes are formulated in compositions for administration to the subject. In some embodiments, the composition is a pharmaceutical composition. In some embodiments, the composition further comprises a pharmaceutically acceptable concentrations of salt, buffering agents, preservatives, compatible carriers, and and/or other (i.e., secondary) therapeutic agents. A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other non-toxic compatible substances employed in pharmaceutical formulations.

To treat the disease/conditions described herein, an effective amount of the MSC exosomes or the composition comprising the MSC exosomes is administered to a subject in need thereof. An “effective amount” is the amount of an agent that achieves the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

In some embodiments, the effective amount is a dosage of an agent that causes no toxicity to the subject. In some embodiments, the effective amount is a dosage of an agent that causes reduced toxicity to the subject. Methods for measuring toxicity are well known in the art (e.g., biopsy/histology of the liver, spleen, and/or kidney; alanine transferase, alkaline phosphatase and bilirubin assays for liver toxicity; and creatinine levels for kidney toxicity).

“Treat” or “treatment” includes, but is not limited to, preventing, reducing, or halting the development of a lung disease, reducing or eliminating the symptoms of lung disease, or preventing lung disease.

A subject shall mean a human or vertebrate animal or mammal including but not limited to a rodent, e.g., a rodent such as a rat or a mouse, dog, cat, horse, cow, pig, sheep, goat, and primate, e.g., monkey. In some embodiments, the subject is a companion animal. “A companion animal,” as used herein, refers to pets and other domestic animals. Non-limiting examples of companion animals include dogs and cats; livestock such as horses, cattle, pigs, sheep, and goats; and other animals such as mice, rats, guinea pigs, and hamsters.

In some embodiments, the subject is a female subject. In some embodiments, the subject is a fetus. In some embodiments, the subject is a female human subject. In some embodiments, the subject is a human fetus.

The subjects may be those that have a disease described herein amenable to treatment using the exosomes described in this disclosure, or they may be those that are at risk of developing such a disease. The methods of the present disclosure are useful for treating a subject in need thereof. A subject in need thereof can be a female subject having or is at risk of infertility, a female subject who has or is at risk of developing placental insufficiency, or a fetus that is suffering from fetal growth restriction. The present disclosure further contemplates administration of the MSC exosomes even in the absence of symptoms indicative of a disease or disorder as described herein.

In some embodiments, the MSC exosome or the composition comprising the exosome is administered to a subject (e.g., a female subject or a fetus) once. In some embodiments, repeated administration of the MSC exosomes, including two, three, four, five or more administrations of the MSC exosomes, is contemplated. In some instances, the MSC exosomes may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ.

The MSC exosomes may be administered by any route that effects delivery to the uterus and/or the fetus. For administering the MSC exosomes to the female subject, systemic administration routes such as intravenous injection or continuous infusion are suitable. In some embodiments, the MSC exosomes are administered via intrauterine injection. In some embodiments, for administering the MSC exosome to the fetus, the MSC exosomes may be administered to the pregnant female subject and indirectly delivered to the fetus. For example, the MSC exosomes may be intravenously injected to the pregnant female subject, be injected to the uterus of the pregnant female, be injected to the ammonic fluid of the pregnant female subject, or via injection into the umbilical vein of the umbilical cord (done routinely to give blood transfusions to anemic fetuses from Rh disease that manifest significant hemolysis). One skilled in the art is able to choose the suitable routes of administration.

The MSC exosomes, may be formulated for parenteral administration by injection, including for example by bolus injection or continuous infusion. Formulations for injection may be presented in unit dosage form, e.g., in ampoules or in multi-dose containers, with or without an added preservative. The compositions may take such forms as water-soluble suspensions, solutions or emulsions in oily or aqueous vehicles, and may contain formulatory agents such as suspending, stabilizing and/or dispersing agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase solubility. Alternatively, the exosomes may be in lyophilized or other powder or solid form for constitution with a suitable vehicle, e.g., sterile pyrogen-free water, before use.

In some embodiments, for treating placental insufficiency in a female subject, the MSC exosome is administered antepartum (before the delivery of the fetus). In some embodiments, the MSC exosome is administered in the first, second, and/or third trimester. In some embodiments, administering the MSC exosomes early during pregnancy (e.g., in early second trimester or first trimester) to female subjects that are at risk of placental insufficiency may reduce the likelihood of complications in the fetus (e.g., fetal growth restrictions and/or fetal loss). In some embodiments, the MSC exosome is administered intrapartum (during the act of birth).

In some embodiments, for treating fetal growth restriction, the MSC exosome is administered antenatal (before the fetus is born). The MSC exosome may be administered at any gestation age. In some embodiments, the MSC exosome is administered intrapartum (during the act of birth). In some embodiments, the MSC exosome is administered perinatal (time period immediately before or after birth, e.g., 4 weeks, 3 weeks, 2 weeks, 1 week, 1 day, or 1 hour before or after birth).

In some embodiments, other agents suitable for treating the conditions/diseases described herein are used in combination with the MSC exosomes for the treatment of the conditions/diseases. It is to be understood that other agents to be administered to subjects being treated according to the disclosure may be administered by any suitable route including oral administration, intranasal administration, intratracheal administration, inhalation, intravenous administration, etc. Those of ordinary skill in the art will know the customary routes of administration for such secondary agents.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1 Mesenchymal Stromal Cell-Derived Exosome for Use in Antenatal Therapy

The goal of the present work is to investigate the therapeutic properties of mesenchymal stromal cell-derived exosomes for the treatment of pregnancy related conditions that have downstream effects on neonatal health. To date, much of the research on neonatal disease has centered on the contribution of post-natal insults. However, emerging evidence suggests that placental insufficiency primes the developing fetus further injury from post-natal exposures and is associated with increased rates of disease, such as neonatal lung disease [1, 2]. In line with this perspective, the present research focuses on the role of preeclampsia in fetal health and development. In preeclamptic pregnancies, alterations in the uterine immune environment lead to abnormal placentation, intrauterine inflammation and fetal growth restriction [3]. This pathological, proinflammatory intrauterine environment may cause a primary insult in the developing fetus post-natal damage. Preeclamptic disease and its complications in the fetus/neonate remain highly difficult to treat despite a variety of attempted interventions. Thus the utility of cell-based treatments has become an emerging area of research for therapeutic intervention.

Fetal growth restriction is a significant global health problem with an increasing impact on fetal morbidity and mortality world-wide. Each year, an estimated 23 million growth-restricted infants are born in developing countries (approximately 20% of live births), and growth restriction puts both full term and preterm infants at increased risk for mortality [4]. Growth restriction has multi-system effects with long term impacts on fetal health, particularly in the developing lung. IUGR infants have an overall higher incidence of bronchopulmonary dysplasia (BPD) with the combination of extreme prematurity and growth restriction putting infants at the highest risk for BPD [5, 6]. Further, preeclampsia itself has also been significantly implicated in BPD risk [7]. Thus, preeclamptic disease and its significant causality in fetal growth restriction have long term impacts for newborn health.

While the disease of preeclampsia is heterogeneous with a multifactorial pathogenesis, a subset of early onset, severe preeclamptic pregnancies involve alterations in the uterine immune environment lead to abnormal placentation, intrauterine inflammation and fetal growth restriction [8]. This pathological, proinflammatory intrauterine environment may cause a primary insult in the developing fetus post-natal damage. Due to its multi-factorial etiologies, preeclampsia remains highly difficult to treat despite a variety of attempted interventions [3]. Pharmacologic treatment can attenuate some maternal symptoms, but no medications to date have been able to mitigate the fetal consequences of this disease. Modulation of the intrauterine environment through biologic therapies may be an important mechanism by which fetal growth restriction can be addressed within preeclampsia. As the source of preeclamptic disease resides primarily within the placenta, targeting therapies that influence the placental interface can have significant benefits for both mother and infant.

Mesenchymal stromal cells (MSC) are well-characterized for their ability to ameliorate a variety of disease processes [9]. These cells have pluripotent capabilities and are involved in tissue homeostasis through cell-cell interactions and secretion of soluble factors. Though MSCs can migrate to injured tissues, they have limited ability for long-term engraftment/expansion. It has been demonstrated that soluble mediators derived from MSCs can equally convey their therapeutic effects [9-11]. Of particular interest are MSC-derived exosomes, a subset of secreted membrane-bound extracellular vesicles (EV). Exosomes, which are EV of 30-150 nm size, contain a variety of surface proteins and cargo including immunomodulatory proteins, cytokines, messenger RNAs, and microRNAs [9]. MSC-derived exosomes (MEX) have anti-inflammatory and immunomodulatory capabilities but low immunogenic potential, which makes them a particularly interesting therapy for immune-mediated diseases [12]. As the pathogenesis of preeclampsia involves alterations in the intrauterine immune environment [8], MEX may be a novel immunomodulatory therapy for this disease and its sequelae.

It has been hypothesized that preeclampsia-associated placental insufficiency and inflammation cause a harmful environment for the developing fetus. MEX can ameliorate the intrauterine environment during pregnancy through immunomodulatory pathways, thereby improving pregnancy outcomes, fetal growth restriction and fetal health.

The main objectives of this study were two-fold: to evaluate the influence of preeclamptic fetal growth restriction on lung development and to test the therapeutic capacity of MEx on maternal preeclamptic stigmata and fetal sequelae. To explore the therapeutic potential of MEx in preeclamptic pregnancy, the heme-oxygenase 1 (HO-1) was investigated in knockout mouse model [5]. HO-1 is primarily involved in heme degradation producing carbon monoxide, iron and biliverdin. As a stress-inducible enzyme, HO-1 is also an important mediator of immune homeostasis. HO-1 expression is stimulated by inflammatory signals, and it functions to modulate immune activity, particularly in macrophages. Indeed, investigation of HO-1 deficiency in various disease models has identified a central role of HO-1 in macrophage polarization, directing their phenotype towards an anti-inflammatory state [13]. The absence of HO-1 during murine pregnancy has a phenotype of fetal loss, fetal growth restriction with maternal preeclamptic features [14]. Similar to humans, the preeclamptic phenotype of HO-1−/− pregnancy appears to have a multi-factorial etiology, with evidence of systemic vascular changes, alterations in key placental immune populations and increased intrauterine inflammation [15].

To address this hypothesis, a pre-clinical model of preeclampsia/fetal growth restriction has been established using the heme-oxygenase-1 (HO-1) knockout mouse. Heme oxygenase-1 is an enzyme involved in heme degradation with well-characterized concomitant immunoregulatory functions, particularly for macrophages [13]. In pregnancy, HO-1 null (HO-1^{-/-}) female mice exhibit fetal loss as well as maternal preeclamptic-like features of hypertension, proteinuria and fetal growth restriction [14].

HO-1^{-/-} pregnant females exhibit significant fetal loss and growth restriction when compared to wild type pregnancies. Maternal renal pathology has also been identified in the HO-1^{-/-} pregnant females with preeclamptic-like glomerular changes that have been previously described in the HO-1 null pregnancy model [15]. Further, the placental interface in HO-1^{-/-} pregnancies contains significantly higher populations of macrophages with pro-inflammatory phenotypes when compared to wild type pregnancies. To this end, the HO-1^{-/-} mice are an ideal model to explore how maternal macrophage dysregulation in preeclampsia contributes to growth restriction. This model is also a valuable phenotype for the investigation of maternally administered MEX therapy in pregnancy. MEX convey their therapeutic effects, at least in part, through macrophage modulation, as recently shown with their ability to ameliorate experimental bronchopulmonary dysplasia (BPD) [5]. The effects of maternally-administered antenatal MEX therapy on pregnancy loss, fetal growth and intrauterine immune homeostasis are currently being investigated, as well as systemic effects on maternal preeclampsia.

In preliminary studies, it has been found that maternal antenatal treatment with MEX can mitigate fetal loss and growth restriction in the HO-1^{-/-} pregnancy model. Evaluation of the utero-placental immunological repertoire and in vitro embryonic lung/amniotic fluid co-cultures further indicated that maternally administered MEx may alter the intrauterine developmental niche to improve fetal lung development in preeclamptic pregnancies. A concomitant reversal of systemic preeclamptic symptoms has also been identified, namely renal pathology in HO-1^{-/-} pregnant mothers following antenatal MEX treatments. As the origins of neonatal disease are inherently tied to the antenatal intrauterine environment, these findings have significant implications for the therapeutic potential of MEX for maternal and fetal health in pregnancy and far beyond.

### Methods

MEX isolation: MSC and MEX were isolated using an established protocol [16]. Briefly, MSCs were isolated from term healthy umbilical cord Wharton's jelly using a modified in vitro explant culture technique. Cell culture supernatants were collected and subjected to differential centrifugation and exosome isolation by flotation on an OptiPrep (iodixanol) cushion (Sigma) or by size-exclusion chromatography. Isolated exosomal content was then confirmed by western-blot evaluation of exosome-specific expression of CD9, CD63 & Flotillin expression [54].

Timed pregnancies and MEX treatment: Timed pregnancies of HO-1^{+/+} (WT) and HO-1^{-/-} (KO) mice were conducted by the breeding of homozygous male and female pairs with the detection of a vaginal plug as gestational (gd) 0. A bolus dose of purified MEX 5×10⁶ cell equivalents were then administered via tail vein injection at gd 1. This MEX dose for has been previously established in the lab as capable of conferring therapeutic effects in an adult murine model of pulmonary hypertension [55]. For the current study, experimental groups consisted of the following numbers of pregnant females: WT (n=5), KO (n=4), and KO treated with MEX (KO+MEX) (n=3).

Pregnancy evaluation and tissue collection: On gd 12, pregnant female mice were sacrificed via intraperitoneal pentobarbital injection followed by dissection and removal of gravid uteri. Fetal implantation sites (IS) and resorption sites (RS) were enumerated and recorded for evaluation of pregnancy loss. Then using a modified cesarean section technique, intact fetuses were removed from uterus/fetal membranes followed by measurement of fetal crown rump length. Remaining tissues of the IS (including placenta, decidual tissues and fetal membranes) were then further processed for flow cytometry analysis. Finally, maternal kidneys were harvested and placed into formalin for further histological analysis.

Flow cytometry: For the current study, 3 IS tissues from each pregnant dam were processed for flow cytometry using the following method. IS were subjected enzymatic digestion with collagenase Type IV and DNAse (Worthington). Tissue suspensions were then treated with RBC lysis buffer (Roche) and placed over a 40 uM cell strainer. The cell flow-through was pelleted, washed and stained using fluorescently conjugated antibodies against F4/80, CD11b, CD11c, and CD40 (BioLegend). Samples were then analyzed at the Dana Farber Flow Cytometry core. Cell numbers as well as mean fluorescence intensity of cell populations were quantified using FlowJo software (Treestar).

Histology: Formalin-fixed maternal kidneys subsequently processed by paraffine embedding, sectioning and hematoxalin/eosin at the Harvard Medical School Rodent Histology core facility. Renal tissue was then surveyed via serial 10× images of the renal cortex (5/kidney), followed by comparative analysis of glomerular characteristics between experimental groups.

Statistical analysis: GraphPad Prism software was used for all graphical and statistical analyses. Student's t-test and one-way analysis of variance were used as indicated by number of experimental groups. Significance was set at p<0.05.

### Results

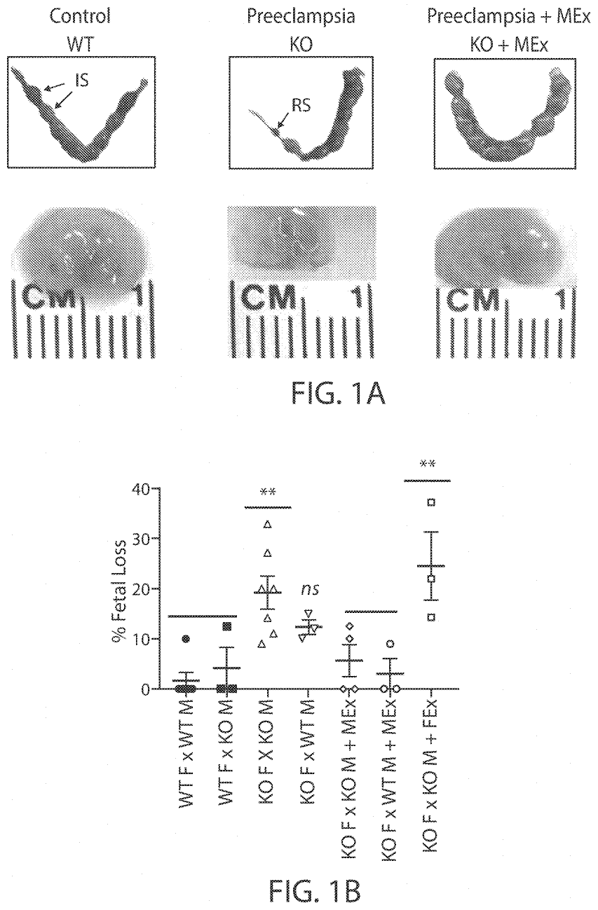

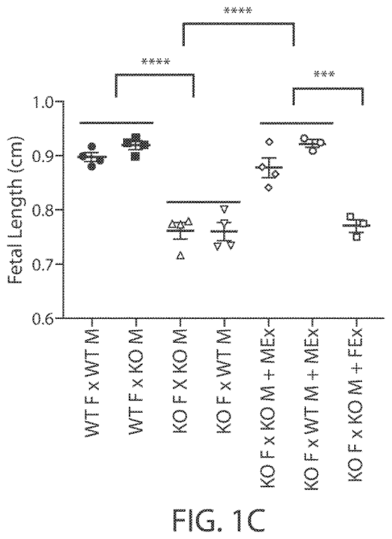

HO-1^{-/-} (KO) pregnant females exhibit significant fetal loss as well as fetal growth restriction at mid-gestation when compared to HO-1^{+/+} (WT) pregnancies (FIGS. 1A to 1D). Following a bolus dose of antenatal MEX administration on gd 1 a significant prevention of fetal loss and growth restriction was identified in KO females (KO+MEX) at mid gestation (gd 12) (FIGS. 1A to 1D).

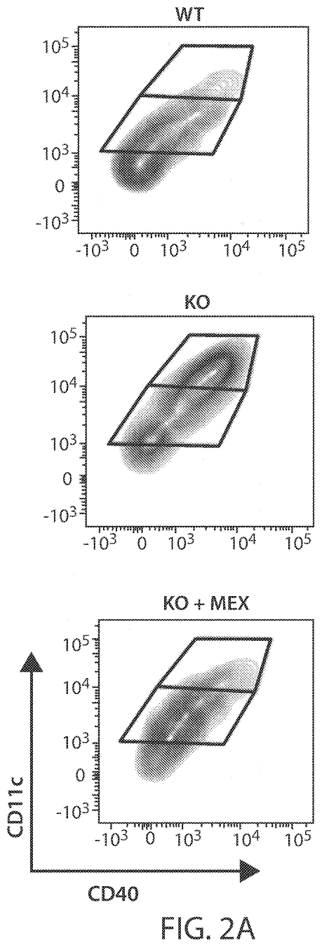

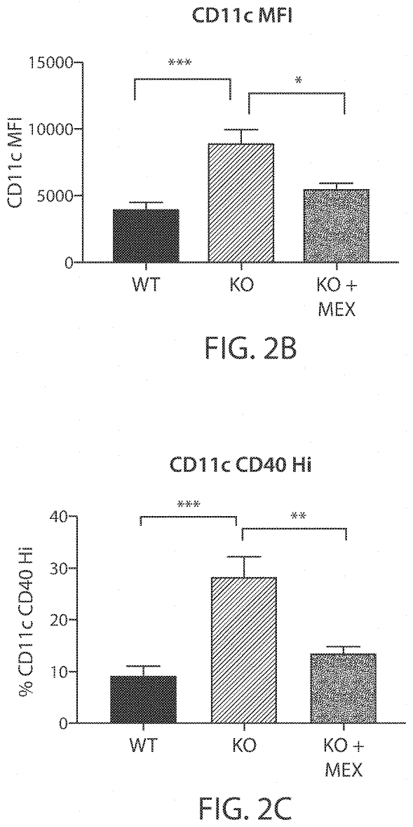

The immune cell populations within fetal implantation sites were further investigated with a particular focus on the macrophage populations as HO-1 is known to be a key regulator macrophage function [13]. Using flow cytometric analysis, a preliminary quantification of macrophages was next performed within mid-gestation implantation sites using uterine macrophage markers [20, 56]. Within this population, a significantly higher percentage of CD11c^{hi}CD40^{hi} cells was detected in KO implantation sites (FIGS. 2A to 2C). As increased expression of CD11c and CD40 are associated with a pro-inflammatory uterine macrophage phenotype [20, 46], KO mice appear have increased infiltration of pro-inflammatory macrophages within the maternal-fetal interface. Following antenatal MEX treatment, a significant decrease was noted in the CD11c^{hi}CD40^{hi} population, both in an evaluation of CD11c fluorescence intensity as well as percentage of CD11c^{hi}CD40^{hi} cells. These findings suggest that antenatal MEX treatment modulates the maternal intrauterine macrophage phenotype, consistent with prior results observed with MEX-mediated macrophage immunomodulation in experimental BPD [5].

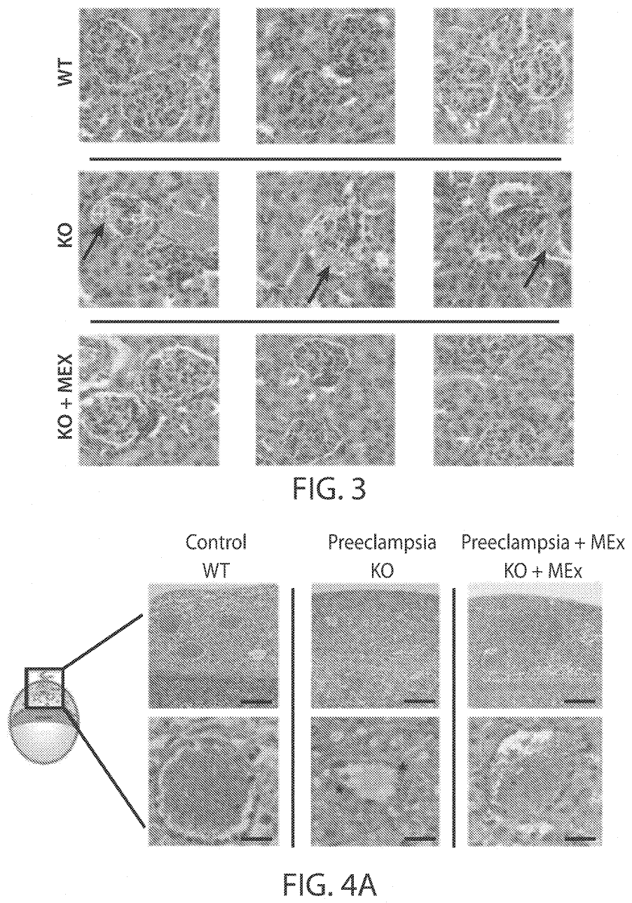

Finally, the KO mothers were evaluated for preliminary signs of preeclampsia at mid-gestation. HO-1^{-/-} pregnant mice are known to exhibit key maternal hallmarks of preeclampsia during pregnancy, including increased systemic hypertension and glomerular architectural changes [15]. As an initial evaluation, maternal renal histology was examined at mid-gestation. In evaluation of renal histology from KO as compared WT mothers, areas of glomerular disruption were identified in the KO maternal kidneys with hallmarks of protein deposition (FIG. 3), which is congruent with glomerular changes in both murine and rat preeclamptic models [15, 16]. Interestingly this glomerular phenotype was reversed following antenatal treatment with MEX (FIG. 3). While further investigations are underway to evaluate other aspects of maternal preeclamptic symptoms (e.g., proteinuria, hypertension), the identification of this maternal KO renal phenotype and its reversal following MEX treatment are an additional indicator of the therapeutic potential of MEX in pregnancy disorders such as preeclampsia.

### Implications

Based on the findings in the HO-1 pregnancy model, it is proposed that MEX treatment can be used for maternal treatment at various time points in the perinatal period for a variety of disease processes. In the model system, antenatal MEX were delivered intravenously but given that MEX are derived from human umbilical cord MSC (a native cell population within the intrauterine environment), this therapy also has the potential to be tested as an intraamniotic therapy during pregnancy. Regarding time of administration, antenatal as well as intrapartum MEX treatments could confer preventative as well as reversal therapeutic aspects, depending on the pregnancy pathology. Indeed, recent studies of murine and rat models testing the effects of maternally-administered whole MSC on pregnancy loss and preeclampsia showed beneficial effects conferred at administration in early first trimester [41] and mid-pregnancy [42], but no studies to date have been published on the isolated effects of MSC-derived exosomes as an antenatal preventative therapeutic modality. Finally, types of disease processes that could be addressed by this therapy include both maternal and fetal conditions. For peripartum maternal conditions, the results suggest that MEX therapy has the potential to be used as an adjunct treatment for infertility, with particular implications for high-risk women seeking IVF treatment. Indeed, previous studies have investigated the potential effects of MSC-secreted products on in vitro based assays showing improvement in ovarian cell maturation as well as embryo optimization [39, 40]. However, the present study is the first known to characterize in vivo effects of maternally administered MEX on pregnancy loss. Additionally, for pregnancy pathologies, given the promising preliminary results in the HO-1^{-/-} pregnancy model, MEX is proposed to have significant therapeutic potential as both a preventative and possibly reversal treatment modality for maternal preeclampsia and its sequelae of fetal growth restriction and premature birth. Overall, the present ongoing studies are addressing important questions on the placental origins of neonatal disease and highlight the highly innovative potential of a maternally-delivered stem cell-based therapy to ameliorate the intrauterine environment for improvement of maternal and fetal health.

### Impact of Maternal Uterine Environment on Offspring Lung Phenotype

It has recently been recognized that the maternal intrauterine environment affects the developing fetal lung. Clinical evidence has shown that pregnancy pathologies that lead to intrauterine growth restriction are associated with increased rates of neonatal lung disease. it was hypothesized that the abnormal intrauterine environment (e.g. as resulting from inflammation, decreased vascular supply, hypoxia) causes a primary insult in the developing fetal lung, predisposing it to further postnatal damage. In the preclinical model of preeclampsia-associated fetal growth restriction using the HO-1 knockout mouse, pups born to either a normal wild type (mWT) or a preeclamptic (HO-1 null, mHO-1−/−) mother were evaluated, which isolates the maternal environment during pregnancy as the primary experimental difference between groups. Using an experimental model of bronchopulmonary dysplasia (BPD), neonatal pups were exposed to either normoxia or hyperoxia (75% oxygen) for 14 days followed by analysis of lung alveolar and airway morphology. The results show that even in normoxia, lungs from pups born to mHO-1 −/− mothers showed signs of alveolar dysplasia with significantly higher mean liner intercept (L(m)) values as compared to pups from mWT (average L(m):mWT 20.6 vs. mHO-1−/− 24.3, p=0.0023, n=8/group). This difference was further amplified following exposure to hyperoxia, in which the lungs of pups born to mHO-1−/− exhibited more severe alveolar simplification and emphysema as compared to pups from mWT (average L(m):mWT 34.6 vs mHO-1−/− 39.6, p=0.042, n=4/group). Treatment of the mother with MEx normalized the histologic appearance of the lung architecture of the mHO-1−/− pups as found in mWT progeny.

### Example 2 Beneficial Effects for Placental Function and Neonatal Lung Development

It has been found that antenatal MSC-exosome (MEx) treatment normalizes key aspects of pre-eclampsia related placental pathology. Additionally, studies have been conducted on the post-natal effects of this treatment on the neonatal lung and have shown that maternal MEx therapy administered throughout pregnancy ameliorates pre-eclampsia-related alterations in fetal lung development, as evidenced by both histological and molecular changes.

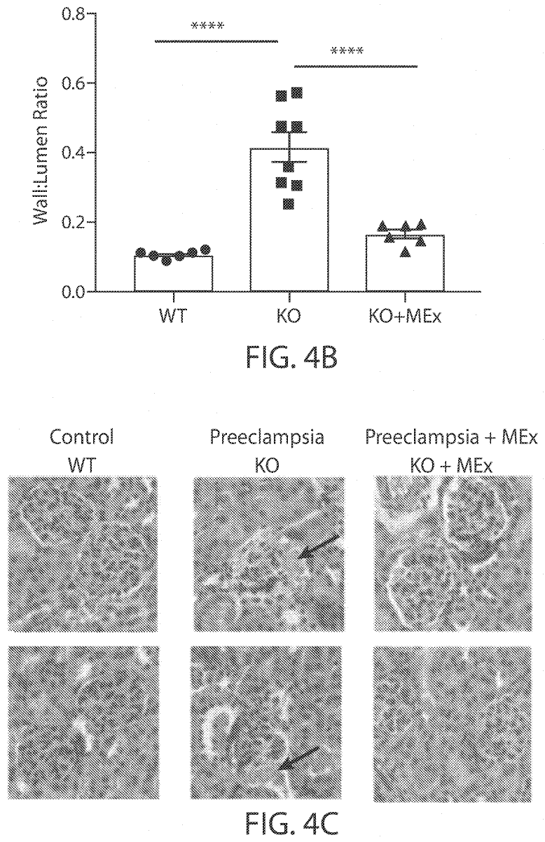

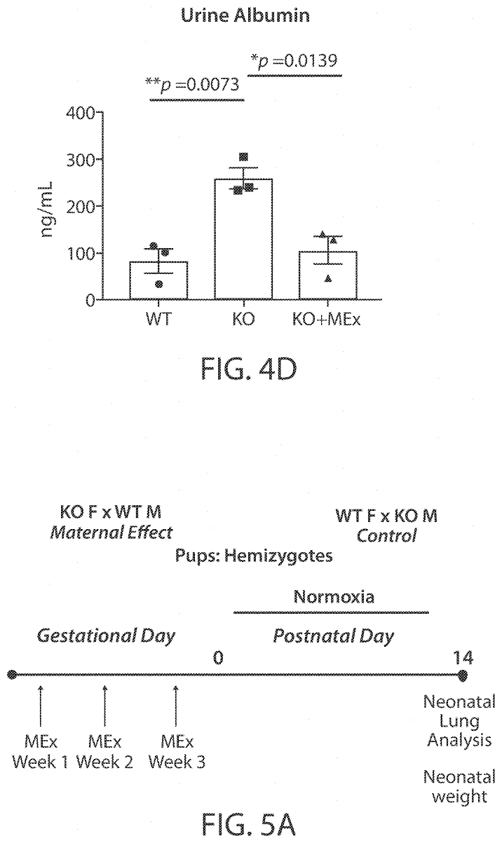

One of the central hallmarks of preeclamptic physiology is the alteration of maternal uterine blood vessels that provide oxygen and nutrients to the developing fetus throughout pregnancy. As the placenta develops in both human and rodent species, the maternal uterine arteries are modified from small-lumen vessels with thick layer of outer smooth muscle to larger conduit, thin-walled vessels. This remodeling is thought to be driven primarily by cytokines released from the resident uterine/placental immune cells, primarily macrophages and natural killer cells [8]. In preeclampsia, the remodeling of maternal blood vessels is significantly reduced, characterized by the sustained phenotype of small-lumen, thick-walled vessels and significantly reducing the nutrient delivery to the fetus. In the present model of preeclampsia, using the heme-oxygenase null mouse (KO), a lack of uterine artery remodeling within the utero-placental interface was detected as compared to wild-type (WT) pregnancies (FIG. 4A). Interestingly, in MEx treated females (KO+MEx), uterine artery morphology was normalized, restoring the large-lumen, thin walled phenotype seen in the wild-type pregnancies (FIG. 4A). Quantification of vessel wall thickness/lumen ratio showed statistical significance for each of these observations (FIG. 4B).

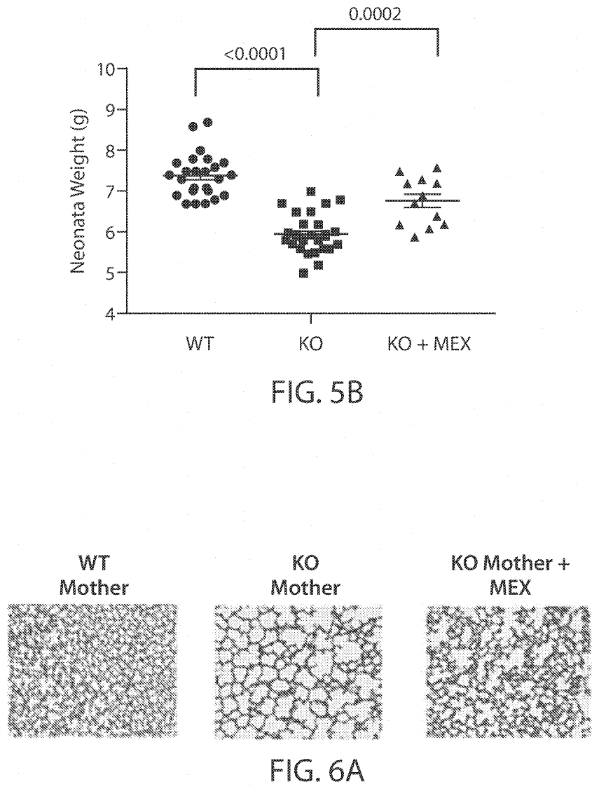

Given the growing attention in maternal preeclampsia associated with worse neonatal lung disease outcomes (discussed in detail within the original submission), the post-natal effects of antenatal MEx treatment on neonatal lung morphology were evaluated. Using a combination of matings, hemizygous, phenotypically equivalent pups were generated, where the only experimental difference was the maternal environment (FIG. 5A). In this series of experiments, a total of three doses of MEx were administered, one during each week of gestation. Following birth, litters were kept in normoxic conditions and their weight and lung tissue was evaluated at post-natal day 14 (FIG. 5A). Comparisons of post-natal weights reflected the findings of fetal growth at mid pregnancy, with smaller pups resulting from the KO maternal environment, which was reversed following maternal MEx treatment (FIG. 5B).

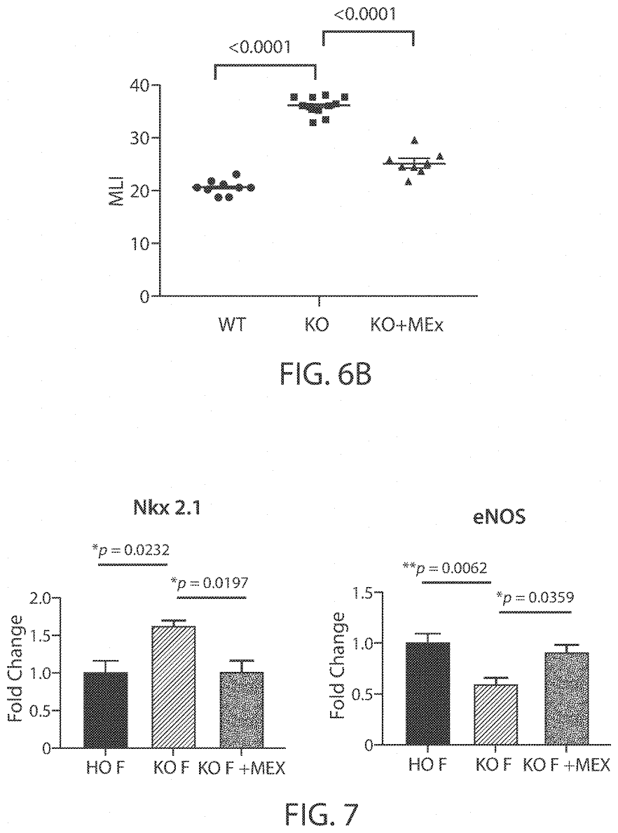

Upon evaluating lung morphology through histological analysis of H&E lung sections, it was found that the pups resulting from the KO maternal environment had increased alveolar simplification, a well-established sign of delayed lung development (FIG. 6A). Quantification of alveolarization using a standardized technique of mean-linear intercept (MLI) analysis showed significant increases in MLI in pups from KO mothers as compared to pups from WT mothers (FIG. 6B). An increase in MLI is evidence of delayed lung development. Further, pups resulting from MEx treated KO mothers showed lung morphology and MLI values similar to that of pups from WT mothers (FIGS. 6A to 6B).

Based on the lung histology findings, fetal lungs were next evaluated for canonical molecular markers of lung development in the respective experimental groups. Using the same model for evaluation of lung development following MEx treatment (FIG. 5A), fetal lung tissue was harvested at gestational d18 and processed for quantitative PCR analysis. Reflecting the results in lung morphology, significant changes were seen in genes Nkx2.1 and eNOS (FIG. 7), both of which are key in the early stages of fetal lung development [32].

These additional data demonstrate that MEx treatment has novel effects on placental morphology which may be the source of MEx reversing fetal loss and growth restriction. Further, it has been discovered that maternal MEx treatment in pregnancy has the ability to confer beneficial effects to the developing fetus, seen by evaluation of multiple parameters: neonatal weight, neonatal lung histology and molecular analysis of lung developmental genes. These new results imply that MEx therapy in pre-eclampsia has systemic, multi-organ effects for the mother. Further, the alteration of the placental morphology and intrauterine environment with this therapy in pregnancy has significant beneficial effects for the developing fetus that are evidenced during developmental and postnatal periods. Additional therapeutic potential for MEx would also now be for pre-eclampsia associated fetal growth restriction and as an antepartum, preventative treatment for neonatal lung disease.

### Example 3 Antenatal Treatment with Mex Ameliorates Preeclamptic Fetal Growth Restriction and Lung Development Through Intrauterine Immunomodulation

In preeclamptic pregnancies, alterations of the uterine immunological milieu can lead to abnormal placentation, release of inflammatory and antiangiogenic factors, and subsequent fetal growth restriction with significant potential to cause a primary insult to the developing fetal lung. Thus, modulation of the maternal intrauterine environment may be a key therapeutic window for the prevention of neonatal lung disease. Using the heme-oxygenase 1 null mouse (HO-1^{-/-}) as a model of preeclampsia, it was demonstrated herein that a preeclamptic intrauterine environment has a significant impact on fetal growth and lung development which is mitigated by maternal treatment with intravenous MEX in early pregnancy. Biodistribution studies show antenatally administered MEX traffic specifically to a subset of cells in the preimplantation uterus. Further, mass cytometric (CyTOF) evaluation of the utero-placental immunological repertoire and lung explant/amniotic fluid co-cultures indicate that maternally administered MEx alters the intrauterine developmental niche to reprogram fetal lung development in preeclamptic pregnancies. Thus, antenatal MEx treatment may provide a highly valuable preventative therapeutic modality for amelioration of preeclamptic physiology and normalization of lung development in preeclamptic disease.

### Results

### Antenatal MEx Therapy Normalizes Preeclamptic Physiology and Fetal Growth Restriction in HO-1−/− Mice

A central hallmark of preeclamptic physiology is the alteration of maternal uterine blood vessels which provide oxygen and nutrients to the developing fetus throughout pregnancy. As the placenta develops in both human and rodent species, the maternal uterine arteries are modified from small-lumen vessels with thick layer of outer smooth muscle to larger conduit, thin-walled vessels. This remodeling is thought to be driven primarily by cytokines released from the resident uterine/placental immune cells, primarily macrophages and natural killer cells. In preeclampsia, the remodeling of maternal blood vessels is significantly reduced, characterized by the sustained phenotype of small-lumen, thick-walled vessels and significantly reducing the nutrient delivery to the fetus. The HO-1−/− pregnant females detected a lack of uterine artery remodeling within the utero-placental interface as compared to wild-type (WT) pregnancies (FIG. 4A). Interestingly, in MEx treated females (KO+MEX), uterine artery morphology was normalized, restoring the large-lumen, thin walled phenotype seen in the wild-type pregnancies (FIG. 4A). Quantification of vessel wall thickness/lumen ratio showed statistical significance for each of these observations (FIG. 4B). Further, the KO mothers were evaluated for other systemic signs of preeclampsia at mid-gestation. HO-1−/− pregnant mice are known to exhibit key maternal hallmarks of preeclampsia during pregnancy, including glomerular architectural changes [15]. In evaluation of renal histology from KO as compared WT mothers, the areas of glomerular disruption were identified and deposition of eosin positive proteinaceous material in the KO maternal kidneys (FIG. 4C), congruent with glomerular changes in both murine and rat preeclamptic models [15, 16]. Further, a significant increase in proteinuria in KO mothers were identified, as assessed by ELISA analysis of urine albumin at mid-pregnancy (FIG. 4D). Interestingly both the glomerular pathology and proteinuria were reversed following antenatal treatment with MEx (FIGS. 4C to 4D).

In examining fetal pathologies, HO-1 −/− (KO) pregnant females exhibit significant fetal loss as well as fetal growth restriction at mid-gestation when compared to HO-1+/+ (WT) pregnancies (FIG. 1A). The maternal contribution to this phenotype was further analyzed by evaluating a combination of homozygous and hemizygous breedings. Significant fetal loss was found exclusively in homozygous KO breedings, which was able to be reversed with MEx therapy (FIG. 1B). However, fetal growth restriction was significantly associated to the maternal KO phenotype, whether the pups resulted from homozygous or hemizygous breedings (FIG. 1C). Antenatal MEx treatment of HO-1−/− females significantly improved fetal growth in all mating types (FIG. 1C). As a control, fibroblast derived exosomes (FEx) had no effect on fetal loss or fetal length (FIGS. 1B to 1C). Overall these data establish canonical symptoms of preeclampsia and significant fetal growth restriction in the HO-1 null pregnancy model, which were significantly ameliorated by MEx therapy.

Biodistribution Studies Highlight MEx Traffic Specifically to the Uterine Interface Following i.v. Administration in Early Pregnancy.

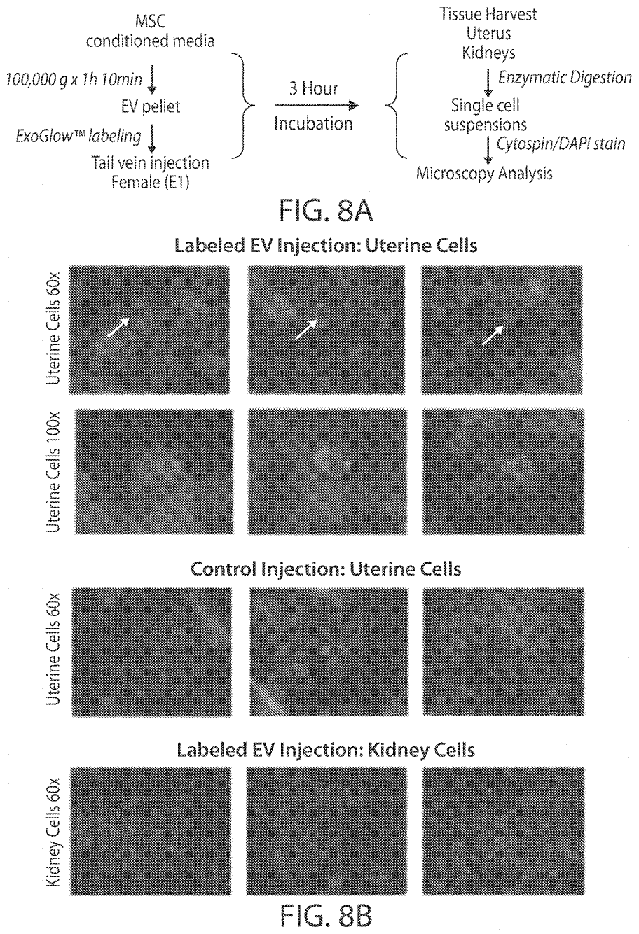

Given the systemic, multi-organ effects seen by maternal MEx administration, it was then studied where these vesicles could be trafficking following tail vein injection in early pregnancy (E1). To accomplish this, extracellular vesicles (EV) were labeled from MSC conditioned media with a membrane specific dye, ExoGlow™ and injected labeled EV into a female mouse at E1 (FIG. 8A). For purposes of visualization, total EV content (which includes MEx) from MSC conditioned media was injected for this analysis. Three hours following injection, uterine and renal tissues were harvested and enzymatically digested to form a single cell suspension followed by microscopic analysis of DAPI-stained cytospins (FIG. 8A). Cytospins from uterine tissues revealed a specific subset of cells positive for uptake of labeled EV (FIG. 8B). Control injections (supernatant of 2nd wash from the labeling procedure) were negative. Further, screening of kidney cell cytospins did not show labeled EV at 3 hours (FIG. 8A). Both tissues were also evaluated 6 hours following injection, and labeled EV were not visualized in either organ at this timepoint.

Taken together, these data suggest that MSC derived EV, which include MEx, injected in early pregnancy are able to traffic to the pre-implantation uterus and are taken up by specific cell types within that tissues. Though additional labeling of cell types with this technique is not possible due to technical limitations of the dye, the frequency of labeled cells within the mixed uterine cell population does suggest a specific population, which may be immune vs parenchymal. Given the known immunomodulatory effects of MEx in other model systems, it was hypothesized that the cells taking up MEx are uterine leukocytes which could be modulated in early pregnancy and confer lasting effects on the intrauterine environment.

### Mass Cytometric (CyTOF) Analysis Highlights Multiple Intrauterine Immune Modifications Conferred by Antenatal MEx Therapy.

In order to understand the means by which MEx alters the HO-1 preeclamptic phenotype, the immune system at the maternal-fetal interface was investigated. Several studies, in pulmonary hypertension and experimental BPD have implicated immunomodulation as a primary mechanism by which MEx confer their therapeutic effects [10, 12]. The immunological landscape of the maternal-fetal interface is comprised of several immune cell types with highly interconnected functions throughout pregnancy [17,18]. Disruption of HO-1 in pregnancy has been associated with changes in multiple immune cell phenotypes, including natural killer cells, macrophages and dendritic cells [15, 19, 20]. Given the interrelated assortment of leukocyte populations at play within the pregnant utero-placental interface, and the varied cells implicated in both human and murine preeclamptic physiology [8], the relative immunologic alterations of MEx therapy in this model system were evaluated by using multi-parameter mass cytometry (CyTOF). This technique, which is a mass-spectrometry based evaluation of single cells labeled with heavy metal tagged antibodies, enables simultaneous analysis of several cell types within a tissue of interest [21]. Using hierarchical clustering algorithms, data can be first be analyzed in an unbiased/unsupervised manner to evaluate the relative abundance of algorithm-identified populations as well as discovery of surface markers altered between experimental conditions. These data can then be combined with supervised analysis of manual gating based on known population markers. In combination, this technique allows both discovery driven and validation approaches to obtain a comprehensive picture of immunological changes within varied experimental conditions based on simultaneous analysis of several cell types.

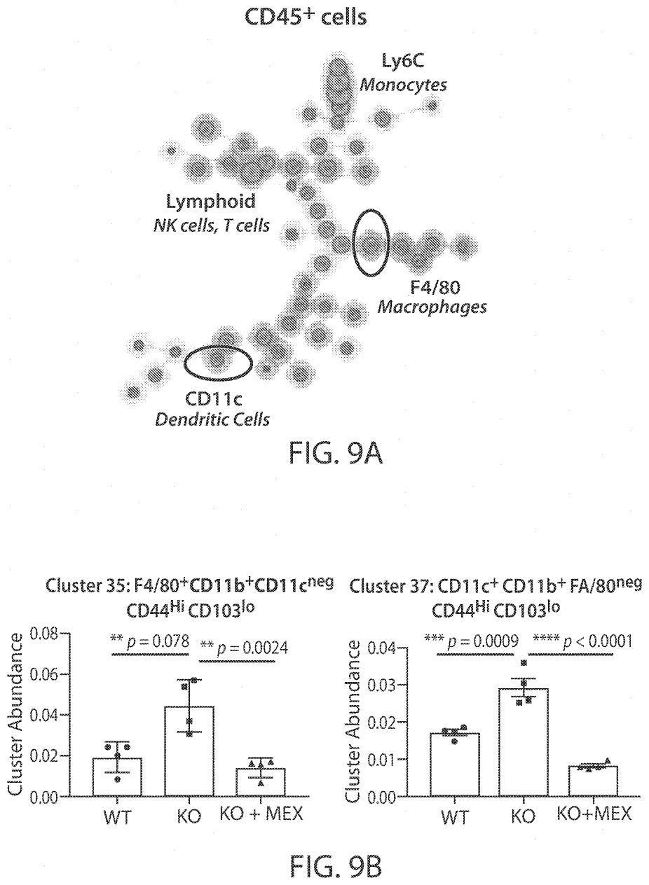

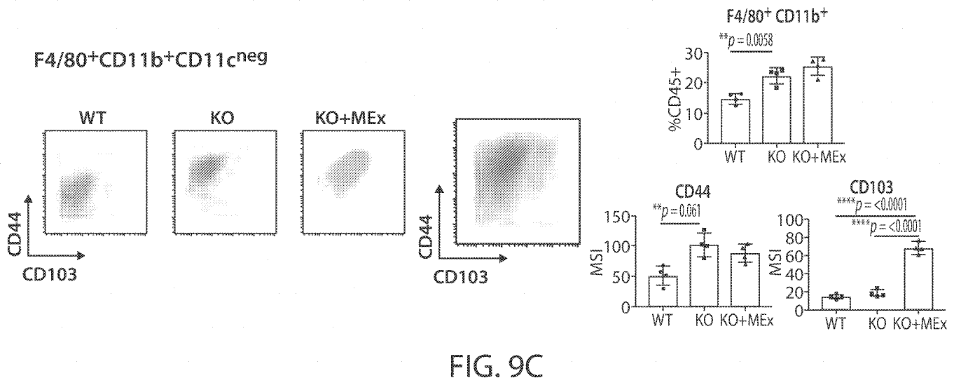

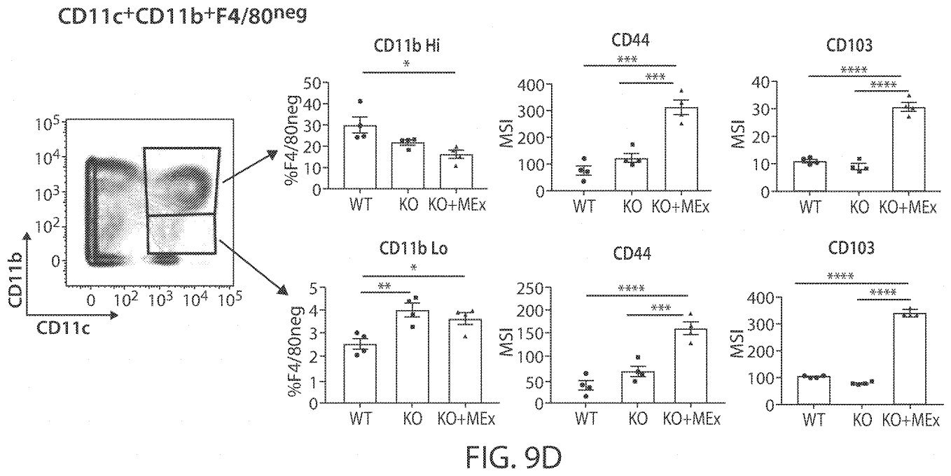

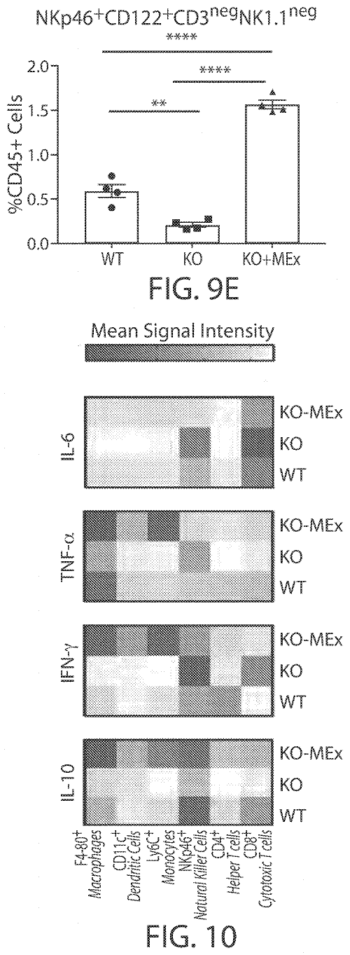

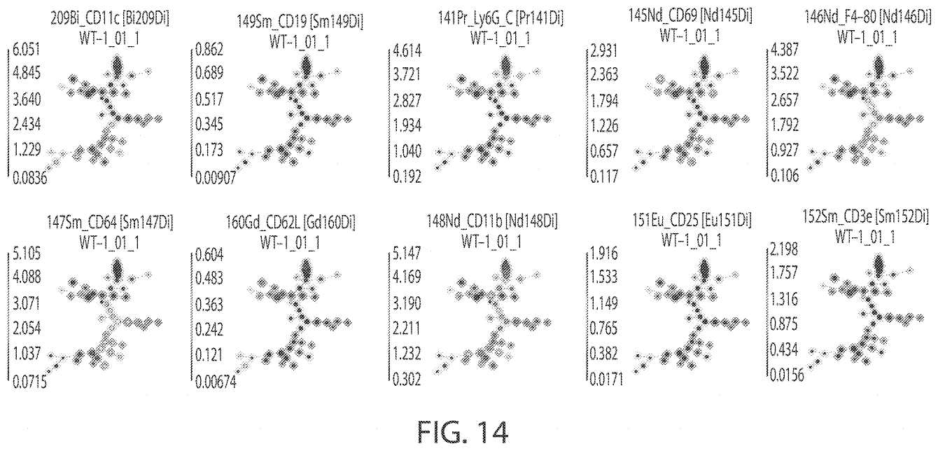

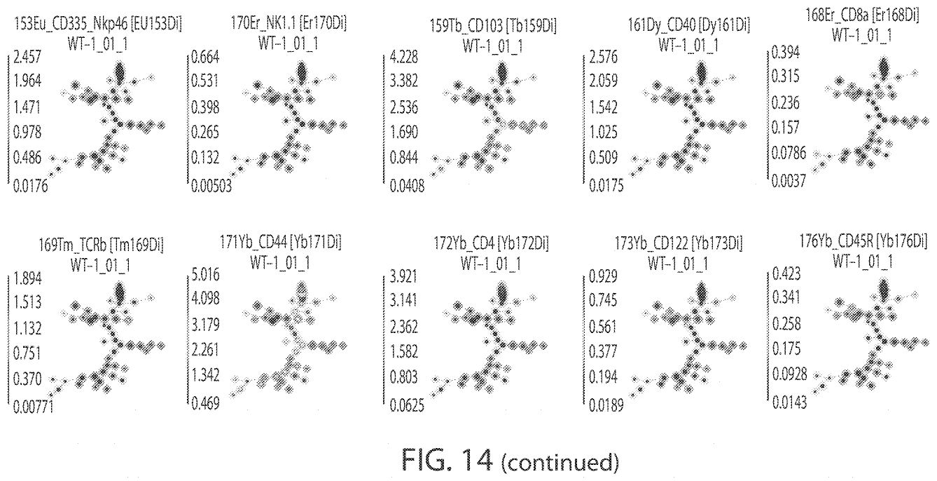

At mid pregnancy (E12) (the timepoint at which multi-organ amelioration of preeclamptic stigmata and fetal growth restriction were observed), the combined uterine, placenta and metrial gland tissues (with fetus removed) were evaluated using CyTOF analysis with a panel of 27 surface/intracellular markers. While the immune populations of each of these tissues are commonly evaluated in separate analyses, the approach with combined tissues was to evaluate the global immune landscape at the utero-placental interface. Cells within these tissues likely function in combination to influence the intrauterine environment during pregnancy. The data was first evaluated using a hierarchical cluster analysis generated from FlowSOM r-script software, which performs significantly well for comparative accuracy/reproducibility in evaluating multi-parameter biological data sets [22]. This analysis generated a cluster map based on relative frequency and intensity of surface markers (FIG. 9A). The identity of each cluster was then visualized using heat maps of surface markers generated by the analysis software (FIG. 14). As a first internal validation, it was noted that the algorithm created both meta-clusters and individual clusters corresponding with established immune populations within the utero-placental interface based on major surface markers (FIG. 9A) [23]. Relative abundance values were then probed for clusters which had significant changes (increase or decrease) in abundance between all three experimental groups. This analysis identified two clusters, 35 and 37 whose abundance values increased significantly within the HO-1−/− pregnancies, and decreased with MEx therapy (FIGS. 9A to 9B). Evaluation of cluster surface marker phenotypes (FIG. 14) revealed two different types of myeloid lineage populations with a particular combination of high CD44 expression and low CD103 expression.

Based on this information, manual gating of these myeloid populations was performed and evaluated for their CD44 and CD103 expression. When evaluating abundance (% CD45+ cells), it was seen that F4/80+CD11b+CD11c neg cells significantly increased in KO pregnancies and remained high in MEx treated dams (FIG. 9C). Further CD11c+F4/80neg cells distributed into CD11bhi/lo groups previously shown for uterine cells with a dendritic cell phenotype [24]. While CD11bhi cells were 10-fold more abundant, their percentages did not change significantly between groups. CD11blo cells were elevated within KO pregnancies and remained high with MEx therapy. Interestingly, in all of these myeloid populations (macrophage and dendritic cell phenotypes), CD44 increased in conjunction with CD103 in MEx treated pregnancies (FIGS. 9C to 9D). The manual gating analysis thus revealed a more specific reason for the changes in abundance of CD44hi/CD103lo populations highlighted by the cluster analysis. With MEx therapy, these myeloid cells are not simply decreasing, their phenotype is being changed from CD103lo to CD103hi. As CD103 surface expression is upregulated with MEx exposure, the abundance of CD103lo cells decreases relative to the CD103 high population that then takes precedence.

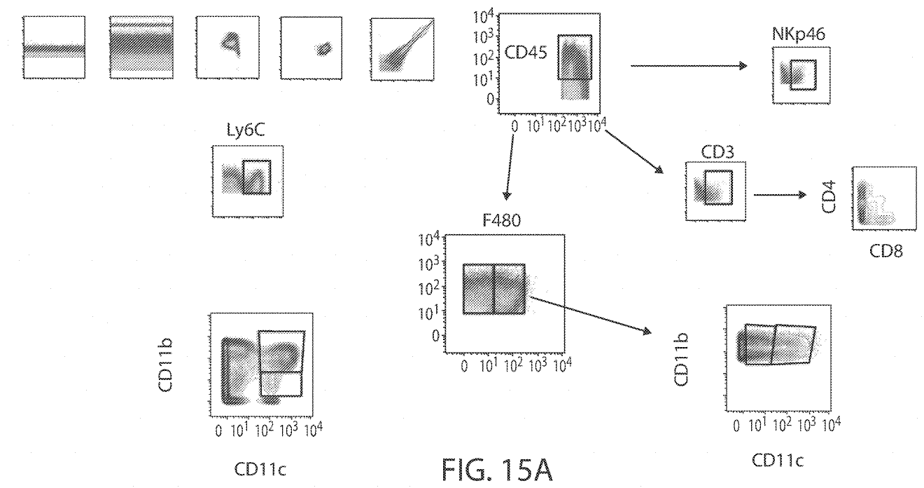

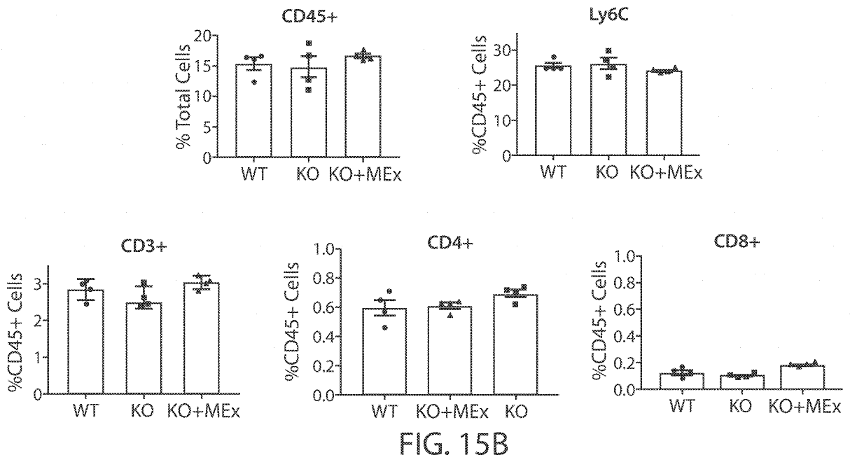

To evaluate the remaining leukocyte populations through supervised analysis of known surface markers, the cells were gated manually to evaluate the relative abundance of all the other major leukocyte populations within the mid-pregnant utero-placental interface (FIGS. 15A to 15B). The only other cell population which showed significant changes between experimental groups were uterine NK (uNK) cells with a phenotype of NKp46+CD122+CD3negNK1.1neg (FIG. 9E). This population has a significant presence within the uterine interface and is unique in its ability to react with Dolichus biflores agglutinin (DBA) [25]. Previous studies in HO-1 null pregnancies have identified histologically that DBA+ uterine NK cells decrease in abundance in the absence of HO-1 null pregnancies [15], a trend which was also found in the uterine NK cell populations. Interestingly, MEx therapy significantly increased the abundance of NK cells within implantation sites (FIG. 9E).

Finally, a comparative analysis of cytokine expression was conducted among each of the major intrauterine immune cell lineages [23]. A specific panel of cytokines significantly associated with both preeclampsia and increased risk of BPD in human and rodent models [26-31] was evaluated, namely intefleukin-10 (IL-10), interferon-gamma (IFN-γ), interleukin-6 (IL-6) and tumor necrosis factor-alpha (TNF-α). Based on comparative analysis of mean signal intensity (MSI), the overall cytokine repertoire was globally changed in KO preeclamptic pregnancies and MEx treatment in preeclamptic-prone KO dams restored an intrauterine cytokine profile similar to that of wild type pregnancy (FIG. 10). Taken together, these data show that preventative MEx treatment in the HO-1 null pregnancy preeclampsia model significantly impacts multiple parameters of the intrauterine immune environment, which may be a key physiologic alteration to promote normalization of pregnancy and fetal growth.

### Preeclampsia-Associated Alterations in Lung Development are Ameliorated by Antenatal Mex Treatment.

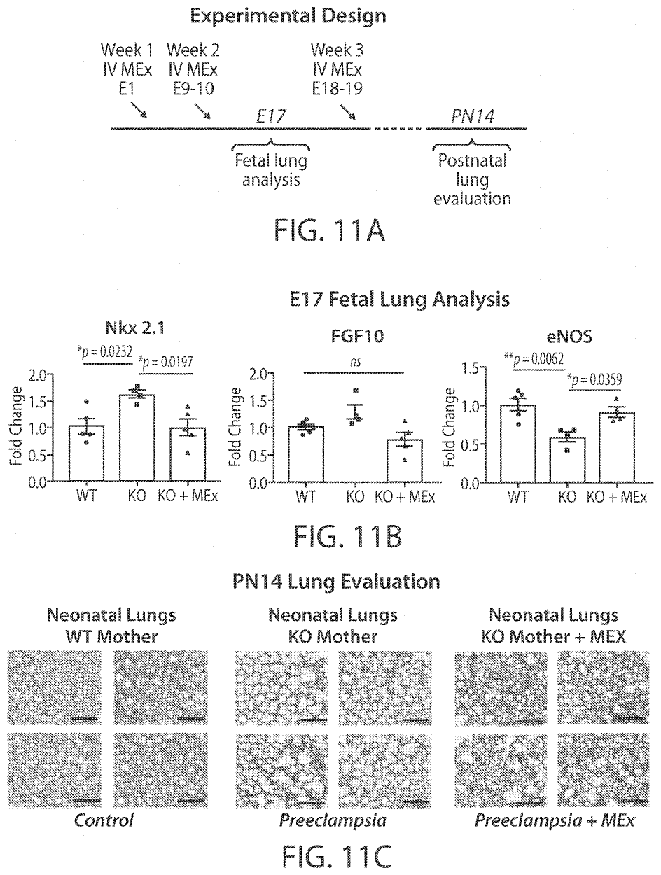

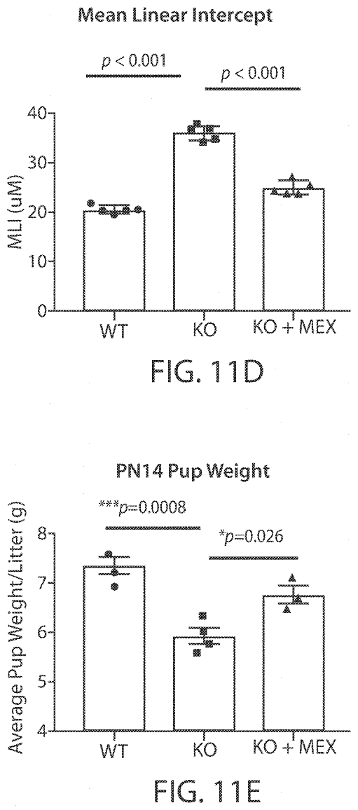

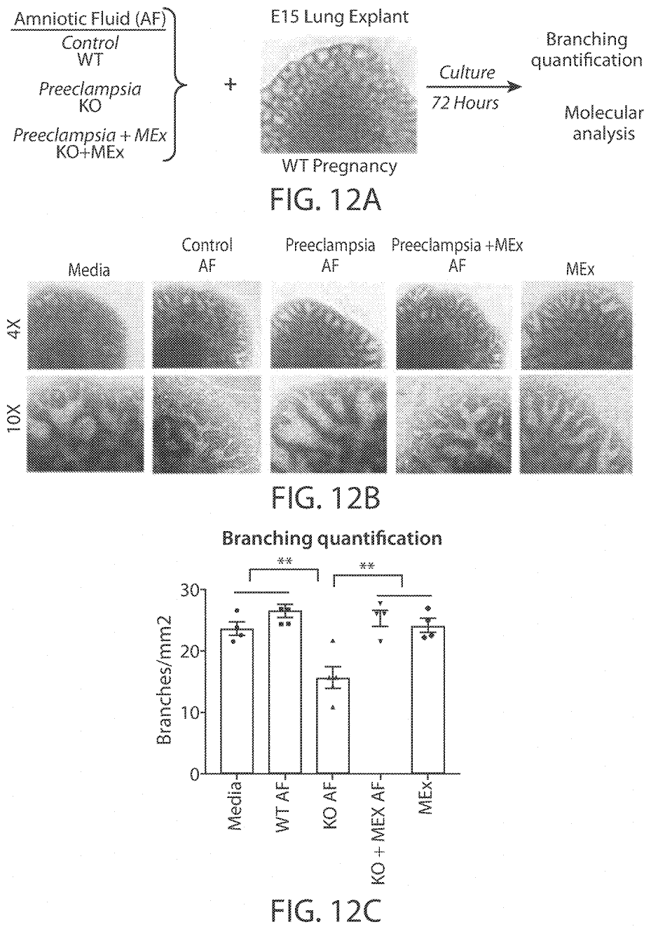

As amelioration of systemic maternal symptoms and fetal growth were observed along with impactful changes in the intrauterine immune environment in this model, it was then evaluated whether antenatal MEx treatment conferred any postnatal effects on the progeny of treated mothers. Focus was given primarily on lung development given the growing recognition of the association between maternal preeclampsia and fetal growth restriction with worse neonatal lung disease outcomes [5-7]. Using a combination of wild type and HO-1−/− matings, hemizygous, genotypically equivalent pups were generated where the main experimental difference was the maternal environment. This allowed us to first evaluate the influence of a preeclamptic maternal environment on fetal lung development and also whether the alterations were observed antenatal MEx treatment in pregnancy had any downstream effects on the fetal lung. In this series of experiments, a total of three doses of MEx were administered, one during each week of gestation (FIG. 11A) and postnatally, equal litter sizes were maintained between all experimental groups, maintaining 7-8 pups/litter.

The fetal lungs were first evaluated for established molecular markers of lung development in the respective experimental groups at E17 (FIG. 11A). At this stage gestation, the fetal lungs are in the canicular stage of development and fetal tissue is formed enough to successfully dissect away from other organs. Following harvest, fetal lung tissue was processed for quantitative PCR analysis and evaluated for developmental genes NKx2.1, fibroblast growth factor (FGF)10 and endothelial nitric oxide synthase (eNOS), which are involved in canonical pathways of alveolarization, branching morphogenesis and pulmonary vascular development [32]. Among those evaluated, only NKx2.1 and eNOS showed significant changes between experimental groups (FIG. 11B). FGF10 did not show significant changes between experimental groups.

It was further evaluated whether these molecular changes observed during fetal development resulted in post-natal changes in lung morphology. Following birth, pups were maintained in normoxic conditions and their weight and lung tissue was evaluated at post-natal day 14 (PN14), an established timepoint at which lung alveolarization can be histologically quantified (FIG. 11C) [10]. Upon evaluating lung morphology, it was found that the pups resulting from the KO maternal environment had disrupted alveolar formation (FIG. 11C). Quantification of alveolarization using mean-linear intercept (MLI) analysis showed significant increases in MLI in pups from KO mothers as compared to pups from WT mothers (FIG. 11D). An increase in MLI reflects decreased alveolar formation, which under normoxic conditions is suggestive of altered lung development/alveolar simplification [33]. Interestingly, pups born from MEx treated KO mothers had a restoration of alveolarization and MLI values similar to that of pups from WT mothers (FIGS. 11C to 11D). Comparisons of pup weight at PN14 also reflected the findings of fetal growth at mid pregnancy, with smaller pups resulting from the KO maternal environment, which was reversed following maternal MEx treatment (FIG. 11E). Taken together, these molecular and histological data at multiple timepoints demonstrate that the maternal preeclamptic environment is associated with altered fetal lung development which can be ameliorated by antenatal MEx therapy.

### Amniotic Fluid Confers the Therapeutic Effect of Antenatal MEx to Improve Fetal Lung Development in Preeclamptic Pregnancies.

Based on changes that were observed in lung development related to MEx modulation of preeclamptic physiology, it was sought to identify particular aspects of the intrauterine environment conferring these effects. Among the biological components within the maternal-fetal interface, amniotic fluid has the most consistent, direct contact with fetal lungs throughout development. In early pregnancy, amniotic fluid is produced as a filtrate of maternal plasma, passing through the fetal membranes by osmotic/hydrostatic forces. In the progression through the second and third trimesters, amniotic fluid contains increasing amounts of fetal components, namely fetal urine and fetal lung fluid with more minor contributions from fetal oral-nasal secretions [34]. Relevant to the current study, increased inflammatory and antiangiogenic factors have been identified within preeclamptic amniotic fluid [35, 36]. Further, a recent report focusing on amniotic influences on lung development in late pregnancy demonstrated that specific intraamniotic exposure of pro-inflammatory LPS and antiangiogenic sFLT within the amniotic compartment are associated with significant changes within the developing lung [33].

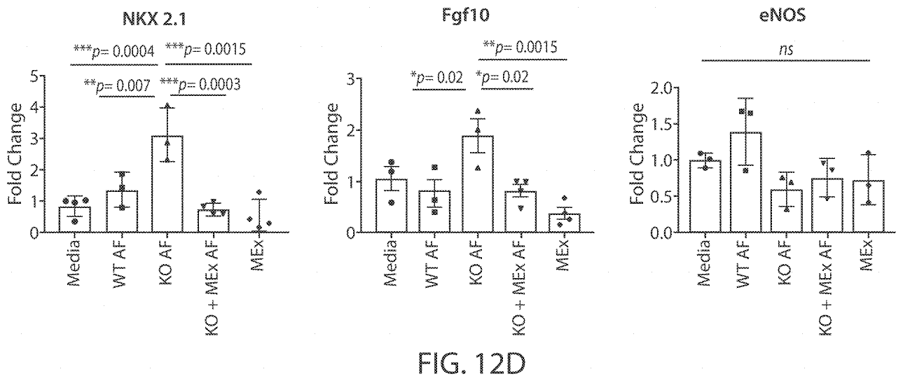

It was thus chosen to evaluate the influence of amniotic fluid on the developing fetal lung with an in vitro co-culture model system utilizing fetal lung explants exposed to various types of amniotic fluid. Lung explants have been previously utilized as a system to demonstrate how exogenous exposures can influence the developing lung, particularly with inflammatory exposures [37]. Lung explants can be assessed visually for branching morphogenesis and harvested following culture for molecular analysis such as qPCR. In this system, E15 lung explants were harvested from fetuses in control WT pregnancies (WT mother) followed by 24 incubation period to allow for adequate attachment and equilibration in transmembrane wells (FIG. 12A). Explants were then exposed to amniotic fluid from control, preeclamptic or Mex-treated preeclamptic pregnancies and MEx alone (FIG. 12A) for a period of 48 hours. Amniotic fluid for these experiments was collected from E12, early second trimester, a targeted timepoint in which the fluid is feasible to obtain from implantation sites and early enough in gestation where the fluid still has a significant component of maternal contents [34]. At the end of the total 72 hour incubation period, explants were imaged via brightfield microscopy and branching was quantified followed by harvest for qPCR analysis of lung developmental genes.

It was first found that media and control (WT) amniotic fluid conditions similar branching values which were also comparable to previously published explants cultured under baseline media conditions at 72 hours (FIGS. 12B to 12C) [38]. Exposure to preeclamptic (KO) amniotic fluid had a significantly decreased number of new branches at this timepoint, suggesting a lack of explant growth during the culture period. Amniotic fluid from MEx treated preeclamptic pregnancies restored the branching morphogenesis to levels similar to control pregnancy. Finally, MEx exposure alone showed branching morphology similar to media and wild type controls, illustrating this direct exposure had no significant changes on explant morphology (FIGS. 12B to 12C). Molecular (qPCR) analysis of mRNA from explants following 72 hours of culture revealed significant upregulation in both NKx2.1 and Fgf10 following exposure to KO amniotic fluid (FIG. 12D), which was reversed in explants exposed to KO+MEx amniotic fluid. Taken together, these in vitro experiments suggest that lung development may be directly impacted by the amniotic fluid environment and alteration of this fluid may be a key interface through which MEx ameliorates preeclamptic alterations in fetal lung development.

### Discussion

The present disclosure, at least in part, demonstrates the association between maternal preeclampsia, fetal growth restriction and increased risk of poor neonatal respiratory outcomes, namely increased risk of BPD. Though detailed characterization of the HO-1−/− preeclamptic environment and mating conditions isolating maternal influence, a link between the intrauterine preeclamptic environment and alterations in fetal lung development was shown which persist into postnatal life. It was further demonstrated that preventative MEx therapy beginning in early pregnancy can significantly ameliorate preeclmaptic physiology in the HO-1−/− model system, resolving maternal symptoms, intrauterine pathology and downstream fetal sequelae.

Based on these findings, it was proposed that MEx treatment can be used for maternal treatment at various timepoints in the perinatal period for amelioration of multiple gestational pathologies including pregnancy loss and the maternal/fetal sequelae of preeclmapsia. For peripartum maternal conditions, the results in reversal of pregnancy loss suggest that MEx therapy could have the potential to be used as an adjunct treatment for infertility, with particular implications for high-risk women seeking IVF treatment. This work highlights the equal importance of uterine optimization in encouraging successful pregnancy, which, in this model, was achieved by maternal MEX administration prior to embryo implantation. Recent studies of murine and rat models testing the effects of maternally-administered whole MSC on pregnancy loss and preeclampsia showed beneficial effects conferred at administration in early first trimester [41] and mid-pregnancy [42], but no studies to date have been published on the isolated effects of MSC-derived exosomes as an antenatal preventative therapeutic modality.

It was also demonstrated that MEX treatment has novel effects on placental morphology which may be the source of MEX reversing fetal loss and growth restriction. Further, it was discovered that maternal MEX treatment in pregnancy has the ability to confer beneficial effects to the developing fetus, seen by evaluation of multiple parameters: neonatal weight, neonatal lung histology and molecular analysis of lung developmental genes. These results imply that MEx therapy in pre-eclampsia has systemic, multi-organ effects for the mother. Further, the alteration of the placental morphology and intrauterine environment with this therapy in pregnancy has significant beneficial effects for the developing fetus that are evidenced during developmental and postnatal periods. Given the sum considerations of the data, it was proposed that MEX has significant therapeutic potential as both a preventative treatment modality for infertility, maternal preeclampsia and its sequelae of fetal growth restriction and lung disease

To address a potential specific source for alteration of the preeclamptic intrauterine environment, the distribution of labeled EV (including MEx) was first visualized from the MSC conditioned media (FIGS. 8A to 8B). The specific uptake within a subset of uterine cells (but not renal cells) suggests a primary location for MEx functionality within the uterine interface. A comprehensive analysis of the immune populations of the utero-placental interface at mid pregnancy (FIGS. 9A to 9E and FIG. 10) was then performed using multi-parameter mass cytometry. These data first give a novel view of the relative abundance and cytokine contributions within normal and experimental preeclamptic conditions. The unsupervised cluster analysis revealed that CD44 and CD103 expression on key myeloid populations were among the most significant changes between the experimental groups. Upon further supervised analysis, it was discovered that MEx therapy significantly increases CD103 expression in CD44hi myeloid populations with both macrophage and dendritic cell repertoire of surface markers. Previous studies have identified CD44 as associated with activation in both macrophage and dendritic cell phenotypes [43, 44]. Further, CD103 is significantly associated with tolerance induction, particularly within populations of intestinal mucosal dendritic cells [45]. CD103 has also been identified on dendritic cell populations in the non-pregnant murine uterus distinguishing between CD11b low and hi populations [24], however the specific role of CD103 in pregnancy and pregnancy-related pathologies has yet to be explored. To date much of the literature on CD103 has identified its critical role in dendritic cell direction of tolerance induction. However, the CyTOF analysis also highlights that following MEx therapy, CD103 induction may also be key in cells with a macrophage surface phenotype and this molecule may be key for immune homeostasis within multiple uterine/placental myeloid populations.

Further supervised analysis of uterine NK cell lineage specific markers also identified significant changes between experimental groups (FIG. 9E) [25]. This data served as an important internal control, linking this study to previously published analyses in HO-1 null pregnancies demonstrating a significant decrease in uterine NK cells in the absence of HO-1 [15]. While not identified in the unsupervised cluster analysis, this data highlights the importance of a combined approach with cluster analysis using unsupervised algorithms in conjunction with gating strategies based on known associative markers. Indeed, a combined approach allows investigators to discover novel marker combinations while also allowing for confirmation of previously identified subpopulations relative to other immune lineages. As the maternal-fetal interface contains a unique repertoire of immune cell lineages working in combination, multi parameter analyses such as mass cytometry will continue to gauge the true relative physiology of these biological interfaces.

Overall, the CyTOF data clarifies two major points regarding the immunology of preeclampsia. It shows that macrophages and dendritic cells are a major relative immunological presence within the mid-pregnant uterus and that their alteration may also be an important component in preeclamptic physiology in the HO-1−/− model. This was not surprising given that HO-1 is most significantly expressed within uterine myeloid populations [20]. Further, MEx therapy are known to significantly alter macrophage phenotypes, which is a primary physiology behind their ability to ameliorate experimental BPD [10]. While much of the focus in previous work in murine and human studies of preeclampsia has focused on the role of NK cells [8], this model system adds to the growing body of literature highlighting the role of uterine/placental macrophages in the pathogenesis of preeclampsia [46]. In the MEx treatment of the HO-1 preeclampsia model, myeloid populations appeared to be significantly altered in conjunction with NK cells. Indeed, macrophages, dendritic cells and NK cells likely work in combination for the establishment and maintenance of pregnancy, likely through an interrelated combination of extracellular signals mediated through cytokine secretion.

The targeted analysis of cytokine production across the major immune populations of the mid-pregnant uterus highlighted alteration in multiple cytokines associated with preeclamptic physiology as well as increased risk of BPD. While more comprehensive cytokine profiling will be required in future studies to evaluate the full spectrum of the intrauterine environment, the data highlight the relative contribution of different cell types in the complex cytokine network of the maternal-fetal interface, which likely shifts dynamically throughout pregnancy and in pregnancy related pathologies. In the model, the HO-1 null preeclamptic pregnant environment was associated with a cytokine profile notably different to that of control pregnancies (FIG. 10), which supports previous analysis [15]. MEx therapy shifted the cytokine profile toward a pattern similar to control/healthy pregnancies (FIG. 10), providing additional evidence of the immunomodulatory capabilities of this treatment in pregnancy.

While these cytokine profiles varied based on cell type and cytokine, some key themes emerge. For example, IL-10 is globally reduced in all cell types in preeclamptic phenotype and increased with MEx therapy (FIG. 10). This is supported in the literature with IL-10 being consistently found to be at lower levels both in preeclamptic placentas [47, 48] and the serum/bronchioalveolar lavage of neonates with BPD [38]. Interestingly, in a 2009 study, IL-10 expression was significantly decreased in a cohort of placental tissues from neonates who went on to develop BPD, suggesting a significant role of IL-10 in the intrauterine determinants of BPD risk [49].

Further, NK cells appear to be secreting higher levels of Interferon gamma, TNF alpha and IL-6 in preeclmapsia which is abrogated in MEx treated preeclamptic pregnancies (FIG. 10). However, myeloid populations show opposite patterns of this cytokine secretion and T cells exhibit a mixed profile depending on their CD4 and CD8 phenotype. Previous data from both preeclamptic studies and evaluations of cytokines in BPD find that up or down regulation of these cytokines can have varying roles in these disease states. For example, in early pregnancy interferon gamma is critical for the establishment of placentation [26]. However, interferon gamma is also persistently elevated in serum of preeclamptic patients [50] and in the bronchiolar lavage of patients with BPD [30]. Overall these data illustrate the complex network of cytokine secretion within the maternal-fetal interface, which likely has shifting contributions from different cell types throughout pregnancy. As therapies for preeclampsia continue to be explored, the goal of maintaining healthy pregnancy should be directed at restoring a global balance of cytokine production, rather than targeting the up or downregulation of an isolated cytokine. Ongoing evaluation utilizing additional markers with multi-parameter cellular analyses will continue to help elucidate the complex interplay of these cytokines during normal and preeclamptic pregnancies as well as their alteration following MEx therapy.

Following characterization of the intrauterine environment, it was then identified that alterations in lung development tied to the preeclamptic maternal environment. The molecular analysis of lung tissue at the canalicular stage showed changes in both NKx2.1 and eNOS in preeclampsia and following MEx treatment, were restored to levels similar to control pregnancy (FIG. 11A). eNOS downregulation in preeclampsia could be a reflection of the antiangiogenic aspects of the preeclamptic environment [51], suggesting future analysis of this model system should also evaluate blood vessel growth within these neonatal lungs.

NKx2.1 upregulation in preeclampsia may represent a more complex physiology. Though this gene is known to be involved in branching morphogenesis [32], the histological analysis showed signs of incomplete alveolar development. Increased NKx2.1 may be an indicator either of a compensatory mechanism of upregulation in response to altered fetal lung development within the preeclamptic intrauterine environment. Alternatively, this upregulation could also be a reflection of global interruption in lung development, suggesting that the preeclamptic-fetal growth restriction is reflecting a developmental delay rather than just a smaller fetus.

Results from in vitro lung explant experiments also support that gene alterations in preeclamptic lung tissue may be a compensatory mechanism for altered fetal branching morphogenesis resulting from the preeclamptic amniotic environment. However, this model system has several limitations. First, it re-creates only a small snapshot of the likely complex interplay of influences on the developing fetal lung and the role of preeclamptic environment in this process. Further these explants are cultured under normoxic conditions (21%), resulting in exposure to relative hyperoxia as compared to that of the intrauterine environment. Finally, as a system assessed without a physiologic blood supply, explants are being assessed in the absence of fetal blood flow coming from the mother/placenta, which likely also has many key factors at play within the developing lung. This may be an explanation of the differential expression of eNOS and FGF10 showed between E17 lungs and ex vivo explant tissues (FIGS. 11A to 11E and FIGS. 12A to 12D), highlighting the likely combined contribution of the amniotic fluid and maternal/placental blood supply in altering fetal lung development during preeclampsia.

However, the benefit of an in vitro lung explant system is that it allows visualization and molecular analysis of a developing lung unit with the majority of lung parenchyma intact. Further, this system allows the evaluation of differing amniotic fluid influences under experimentally controlled conditions, enabling targeted analysis of differences related to changes within the amniotic fluid contents. The key findings of the explant studies were that amniotic fluid from preeclamptic pregnancies caused a decrease in explant branching in addition altered lung developmental gene expression which was reversed in MEx treated preeclamptic pregnancies (FIGS. 12A to 12D). Both NKx2.1 and FGF10 were significantly changed by preeclamptic amniotic fluid in this model system. Of particular note, NKx2.1 gene expression was increased in both E17 lungs in preeclamptic pregnancies and in E15 lung explants exposed to preeclamptic amniotic fluid, and antenatal MEx treatment significantly reduced to levels similar to control pregnancies in both sets of experiments. While both fetal lung analysis and explant data highlight alterations in various developmental genes, the commonality of NKx2.1 suggests this transcription factor and its correlate protein, thyroid transcription factor-1 (TTF-1) may be a key lung developmental pathway altered within the preeclamptic environment. Previous studies on TTF-1 in lung tissues have identified that increased expression of this protein inhibits alveolarization [52]. Further, in histological analysis of lungs from neonates with BPD, TTF expression was increased in regenerating open airways relative to areas of alveolar collapse/inflammation [53].

Finally, the study highlights a key interface for beneficial effects of antenatal MEx therapy resides within alteration of the intrauterine developmental niche, namely alteration of the amniotic fluid contents. The alterations of intrauterine immune populations seen on the CyTOF analysis suggest that modulation of cytokine repertoire being secreted into the amniotic fluid contents themselves could explain these therapeutic effects. As MEx-mediated amelioration of the preeclamptic influence on fetal growth restriction and lung disease is very likely multi-factorial, ongoing analysis evaluating additional timepoints with continued multi-parameter analyses of developmental gene networks and intrauterine contents will help further elucidate the complex network of mechanisms involved in this physiology. Overall, this work provides key evidence for the intrauterine origins of neonatal disease and highlights the highly innovative potential of a maternally-delivered stem cell-based therapy to ameliorate the intrauterine environment for improvement of maternal and fetal health.

### Methods

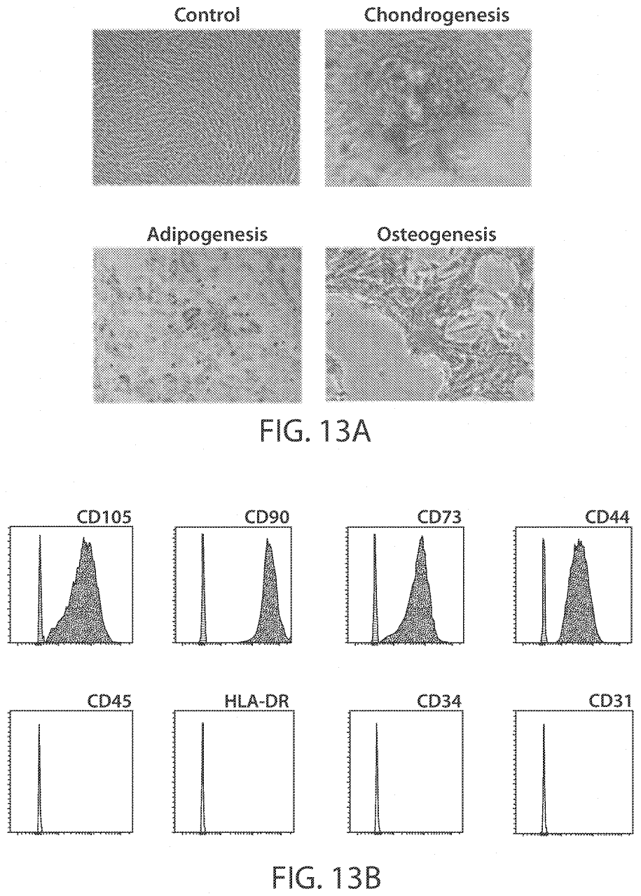

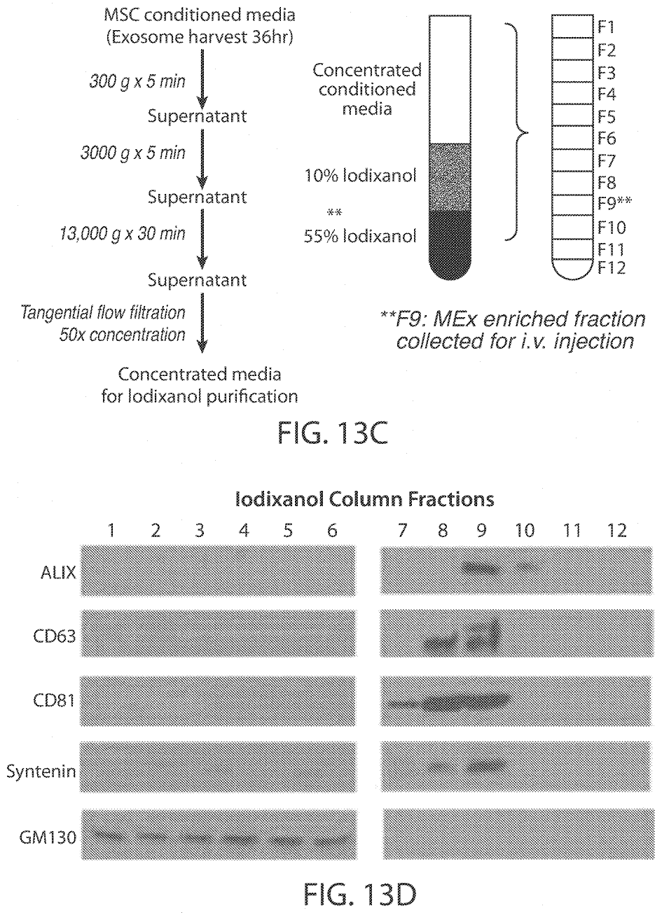

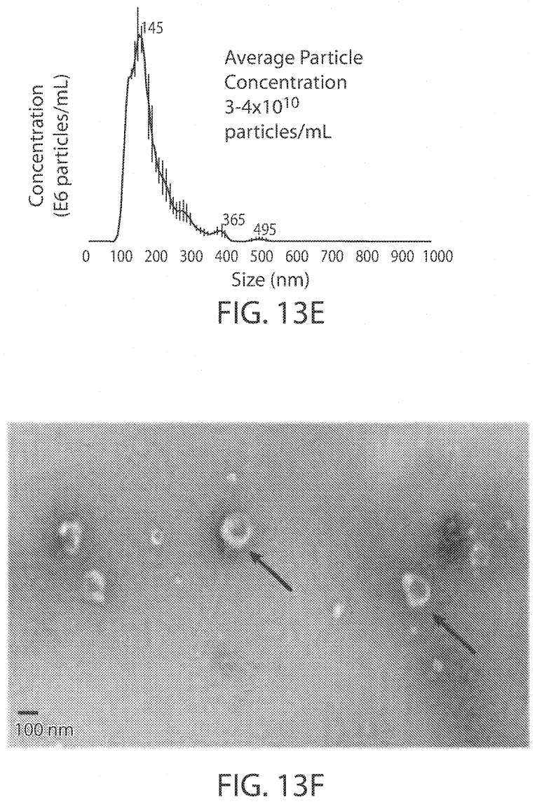

MEx isolation and characterization: MSC and MEx were isolated using an protocol established within the research group (FIGS. 13A to 13F) [54]. Briefly, MSCs were isolated from term healthy umbilical cord Wharton's jelly using a modified in vitro explant culture technique as previously described [10]. Resultant mesenchymal stromal cells were then cultured in α-Modified Eagle Medium (αMEM, Invitrogen) supplemented with 10% fetal bovine serum (Invitrogen), 2 mM L-glutamine and 1% penicillin/streptomycin in p150 dishes (Corning) at 37° C. in a humidified atmosphere with 5% CO2 and allowed to reach a confluency of 60-70% prior to each passage. MSC differentiation potential at passage (P)2 was assessed using differentiation assay kits for chondrogenesis, adipogenesis and osteogenesis, per manufacturer instructions (StemPro, Gibco). Ability to differentiate into these three lineages was used as early confirmation of MSC morphology for each MEx prep (FIG. 13A). MSC purity at P2 was further evaluated via single color flow cytometry (FIG. 13B) using fluorescently conjugated antibodies against human MSC positive markers CD105, CD90, CD73, and CD44 (BD Pharmingen) as well a human negative MSC negative marker panel (BD Pharmingen).

For exosome harvest (FIG. 13C), MSC preps were further cultured to P3, and upon reaching 90% confluency, cells were serum starved for 36 hours followed by collection of cell culture supernatant (conditioned media). This conditioned media was then subjected to differential centrifugation and exosome isolation by flotation on an OptiPrep (iodixanol) cushion (Sigma) (FIG. 13C). Isolated exosomal content in fraction 9 (MEx enriched fraction) was then confirmed by western-blot showing positive expression of exosome-specific surface proteins ALIX, CD63, CD81, and syntenin-1 as well as negative expression of GM130 [54]. (FIG. 13D). Antibodies for western blot analysis were sourced as follows: ALIX (Santa Cruz), CD63 (Sigma-Aldrich), CD81 (Santa Cruz), syntenin-1 (Thermo Fisher), and GM130 (Cell Signaling). Purified MEx were additionally evaluated using NanoSight analysis, to assess particle size distribution/concentration (FIG. 13E) as well as electron microscopy to visualize vesicle morphology and size in each prep (FIG. 13F).

Timed pregnancies and MEx treatment: Timed pregnancies of HO-1+/+(WT) and HO-1 −/− (KO) mice were conducted by the breeding of homozygous male and female pairs with the detection of a vaginal plug as gestational day/embryonic day (E) 0. A bolus dose of purified MEX (5×106 cell equivalents) was then administered via tail vein injection at E1. This MEX dose for has been previously established in the lab as capable of conferring therapeutic effects in an adult murine model of pulmonary hypertension [55].

Pregnancy evaluation and tissue collection: On E12, pregnant female mice were sacrificed via intraperitoneal pentobarbital injection followed by dissection and removal of gravid uteri. Fetal implantation sites (IS) and resorption sites (RS) were enumerated and recorded for evaluation of pregnancy loss. Then using a modified cesarean section technique, intact fetuses were removed from uterus/fetal membranes followed by measurement of fetal crown rump length. Remaining tissues of the IS (including placenta, decidual tissues and fetal membranes) were then further processed for mass cytometry analysis. During dissection of IS tissues, amniotic fluid was collected a sterile culture dish, centrifuged at 3000×g for 10 min at 4 C. The supernatant was then snap frozen for further use in lung explant cultures (see below). Finally, maternal kidneys were harvested and placed into formalin for further histological analysis.

Histology: Formalin-fixed placentas kidneys and neonatal lungs were subsequently processed by paraffin embedding, sectioning and hematoxylin/eosin (H&E) per standard procedures. Maternal spiral artery morphology within placental tissues were analyzed via serial 10× images of metrial gland/placental interface (5/placenta), followed by measurement of artery vessel wall:lumen ratio. Renal tissue was then surveyed via serial 10× images of the renal cortex (5/kidney), followed by comparative analysis of glomerular characteristics between experimental groups. PN14 lungs were perfused with PBS via the right ventricle a constant pressure of 25 cm H20. Lungs were then inflated using formalin endotracheal infusion at 15 cm H2O [10]. Lungs were subsequently processed for H&E paraffin sections as described above. Mean linear intercept lung analysis was calculated from serial 10× lung images taken by two independent investigators, with slides blinded for experimental group analysis.

Urine analysis: At time of sacrifice on E12 (as described above), bladders were exposed and urine was aspirated via bladder puncture with a sterile 1 mL 30 G syringe. Urine samples were subsequently snap frozen and banked at −80° C. for further analysis. Upon collection of full experimental cohort, urine samples were quick thawed and processed for mouse albumin ELISA analysis per manufacturer's instructions (Abcam).

Biodistribution of labeled extracellular vesicles (EV): MSC conditioned media from a total of 12×106 cells was harvested as described above followed by centrifugation at 100,000×g for 1 hour 10 min. Total extracellular vesicles (EV) were then labeled with ExoGlow™ labeling kit per manufacturer's instructions (SystemBio). Labeled EV were then immediately injected into the tail vein of E1 females. Following a 3 hour incubation, both uterine and kidney tissues were harvested and digested with collagenase Type IV and DNAse (Worthington). Tissue suspensions were then treated with RBC lysis buffer (Roche) and placed over a 40 uM cell strainer. The cell flow-through was then pelleted, washed and the resulting single cell suspensions were spun onto charged microscope slides with a cytospin equipment. Slides were dried overnight, cover slipped with Fluorshield/DAPI solution (Invitrogen) and visualized using a Nikon Eclipse 80i microscope (Nikon, Tokyo, Japan).

Mass cytometry: Six pooled IS tissues (fetus removed) from each pregnant dam were processed for mass cytometric analysis using the following method. IS were subjected enzymatic digestion with collagenase Type IV and DNAse (Worthington). Tissue suspensions were then treated with RBC lysis buffer (Roche) and placed over a 40 uM cell strainer. The cell flow-through was pelleted, washed and counted. 0.8-1×106 cells were stained with heavy-metal conjugated primary antibodies targeting a panel of 27 surface and intracellular markers, per manufacturer's protocol (Fluidigm, evaluating 0.8-1×106 cells per animal. Unsupervised, multi-parameter hierarchical cluster analysis was performed using FlowSOM analysis R-script software (Cytobank.org) with individual cluster threshold of 49. Further analysis of population frequency as well as mean signal intensity of cell populations were quantified using FlowJo software (Treestar).

qPCR analysis: Fetal lung tissues harvested at E17 and fetal lung explants after 72 h of culture were snap frozen followed by RNA extraction with Tri-Reagent (Sigma) per manufacturer's protocol. RNA transcripts were subsequently evaluated with Taqman probes/primers (Thermofisher) for the following targets: NKx1.1, FGF10, and eNOS. Target expression was normalized to housekeeping transcript nuclear pore protein 133 (Nup133) and relative expression was quantified via fold change relative to WT using 2−ΔΔCT calculations.

Lung explant co-cultures: Using a stereomicroscope, E15 fetal lungs were harvested via a left thoracotomy and extraction of left lung lobe. Fetal lungs were then dissected into 0.5- to 1-mm3 cubes and placed onto 24-mm clear polyester membrane supports (Transwell, 0.4-μM pore size; Corning, Corning, N.Y.). Serum free DMEM (Thermofisher) was added only to the basal compartment and explants were then placed into a humidified atmosphere of 95% air-5% CO2 at 37° C. Following 24 hours of culture, 100 uL of media containing the following components was added directly onto each explant according to experimental conditions: media only, media+WT amniotic fluid (1:10), media+KO amniotic fluid (1:10), media+KO/MEx amniotic fluid (1:10) or media+MEx (1:10). Brightfield images of explants were acquired at 24 h and 72 h of culture. From images taken at 72 hours, branch tips were visually counted and airway branching was expressed as the number of new branches per mm2 of explant.

Statistical analysis: GraphPad Prism software was used for all graphical and statistical analyses. One-way analysis of variance was used in all statistical analyses between experimental groups. Significance was set at p<0.05.

### REFERENCES

1. Mestan, K. K., et al., Placental pathologic changes of maternal vascular underperfusion in bronchopulmonary dysplasia and pulmonary hypertension. Placenta, 2014. 35(8): p. 570-4.
2. Kunjunju, A. M., et al., Bronchopulmonary dysplasia-associated pulmonary hypertension: clues from placental pathology. J Perinatol, 2017. 37(12): p. 1310-1314.
3. Mol, B. W. J., et al., Pre-eclampsia. Lancet, 2016. 387(10022): p. 999-1011.
4. Lee, A. C., et al., Estimates of burden and consequences of infants born small for gestational age in low and middle income countries with INTERGROWTH-21(st) standard: analysis of CHERG datasets. Bmj, 2017. 358: p. j3677.
5. Lal, M. K., et al., Chronic lung disease of prematurity and intrauterine growth retardation: a population-based study. Pediatrics, 2003. 111(3): p. 483-7.
6. Soudee, S., et al., Fetal growth restriction is worse than extreme prematurity for the developing lung. Neonatology, 2014. 106(4): p. 304-10.
7. Hansen, A. R., et al., Maternal preeclampsia predicts the development of bronchopulmonary dysplasia. J Pediatr, 2010. 156(4): p. 532-6.
8. Laresgoiti-Servitje, E., N. Gomez-Lopez, and D. M. Olson, An immunological insight into the origins of pre-eclampsia. Hum Reprod Update, 2010. 16(5): p. 510-24.
9. Phinney, D. G. and M. F. Pittenger, Concise Review: MSC-Derived Exosomes for Cell-Free Therapy. Stem Cells, 2017. 35(4): p. 851-858.
10. Willis, G. R., et al., Mesenchymal Stromal Cell Exosomes Ameliorate Experimental Bronchopulmonary Dysplasia and Restore Lung Function through Macrophage Immunomodulation. Am J Respir Crit Care Med, 2018. 197(1): p. 104-116.
11. Hansmann, G., et al., Mesenchymal stem cell-mediated reversal of bronchopulmonary dysplasia and associated pulmonary hypertension. Pulm Circ, 2012. 2(2): p. 170-81.
12. Mitsialis, S. A. and S. Kourembanas, Stem cell-based therapies for the newborn lung and brain: Possibilities and challenges. Semin Perinatol, 2016. 40(3): p. 138-51.
13. Naito, Y., T. Takagi, and Y. Higashimura, Heme oxygenase-1 and anti-inflammatory M2 macrophages. Arch Biochem Biophys, 2014. 564: p. 83-8.
14. Ozen, M., et al., Heme oxygenase and the immune system in normal and pathological pregnancies. Front Pharmacol, 2015. 6: p. 84.
15. Zenclussen, M. L., et al., Heme oxygenase-1 is critically involved in placentation, spiral artery remodeling, and blood pressure regulation during murine pregnancy. Front Pharmacol, 2014. 5: p. 291.
16. Li, Z., et al., Recombinant vascular endothelial growth factor 121 attenuates hypertension and improves kidney damage in a rat model of preeclampsia. Hypertension, 2007. 50(4): p. 686-92.
17. Petroff, M. G., Immune interactions at the maternal-fetal interface. J Reprod Immunol, 2005. 68(1-2): p. 1-13.
18. Taglauer, E. S., K. M. Adams Waldorf, and M. G. Petroff, The hidden maternal-fetal interface: events involving the lymphoid organs in maternal-fetal tolerance. Int J Dev Biol, 2010. 54(2-3): p. 421-30.
19. Schumacher, A., et al., Blockage of heme oxygenase-1 abrogates the protective effect of regulatory T cells on murine pregnancy and promotes the maturation of dendritic cells. PLoS One, 2012. 7(8): p. e42301.
20. Zhao, H., et al., Infiltration of myeloid cells in the pregnant uterus is affected by heme oxygenase-1. J Leukoc Biol, 2017. 101(1): p. 217-226.
21. Kimball, A. K., et al., A Beginner's Guide to Analyzing and Visualizing Mass Cytometry Data. J Immunol, 2018. 200(1): p. 3-22.
22. Weber, L. M. and M. D. Robinson, Comparison of clustering methods for high-dimensional single-cell flow and mass cytometry data. Cytometry A, 2016. 89(12): p. 1084-1096.
23. Erlebacher, A., Immunology of the maternal-fetal interface. Annu Rev Immunol, 2013. 31: p. 387-411.
24. Collins, M. K., C. S. Tay, and A. Erlebacher, Dendritic cell entrapment within the pregnant uterus inhibits immune surveillance of the maternal/fetal interface in mice. J Clin Invest, 2009. 119(7): p. 2062-73.
25. Yadi, H., et al., Unique receptor repertoire in mouse uterine NK cells. J Immunol, 2008. 181(9): p. 6140-7.
26. Murphy, S. P., et al., Interferon gamma in successful pregnancies. Biol Reprod, 2009. 80(5): p. 848-59.
27. Lau, S. Y., et al., Tumor necrosis factor-alpha, interleukin-6, and interleukin-10 levels are altered in preeclampsia: a systematic review and meta-analysis. Am J Reprod Immunol, 2013. 70(5): p. 412-27.
28. LaMarca, B. D., et al, Inflammatory cytokines in the pathophysiology of hypertension during preeclampsia. Curr Hypertens Rep, 2007. 9(6): p. 480-5.
29. Koksal, N., et al., Value of serum and bronchoalveolar fluid lavage pro- and anti-inflammatory cytokine levels for predicting bronchopulmonary dysplasia in premature infants. Eur Cytokine Netw, 2012. 23(2): p. 29-35.
30. Ambalavanan, N., et al., Cytokines associated with bronchopulmonary dysplasia or death in extremely low birth weight infants. Pediatrics, 2009. 123(4): p. 1132-41.
31. Aghai, Z. H., et al., IFN-gamma and IP-10 in tracheal aspirates from premature infants: relationship with bronchopulmonary dysplasia. Pediatr Pulmonol, 2013. 48(1): p. 8-13.
32. Herriges, M. and E. E. Morrisey, Lung development: orchestrating the generation and regeneration of a complex organ. Development, 2014. 141(3): p. 502-13.
33. Tang, J. R., et al., Excess soluble vascular endothelial growth factor receptor-1 in amniotic fluid impairs lung growth in rats: linking preeclampsia with bronchopulmonary dysplasia. Am J Physiol Lung Cell Mol Physiol, 2012. 302(1): p. L36-46.
34. Brace, R. A. and C. Y. Cheung, Regulation of amniotic fluid volume: evolving concepts. Adv Exp Med Biol, 2014. 814: p. 49-68.
35. Kupferminc, M. J., et al., Soluble tumor necrosis factor receptors and interleukin-6 levels in patients with severe preeclampsia. Obstet Gynecol, 1996. 88(3): p. 420-7.
36. Vuorela, P., et al., Amniotic fluid—soluble vascular endothelial growth factor receptor-1 in preeclampsia. Obstet Gynecol, 2000. 95(3): p. 353-7.
37. Stouch, A. N., et al., IL-1beta and Inflammasome Activity Link Inflammation to Abnormal Fetal Airway Development. J Immunol, 2016. 196(8): p. 3411-20.
38. Prince, L. S., FGF10 and Human Lung Disease Across the Life Spectrum. Front Genet, 2018. 9: p. 517.
39. Sun, L., et al., Exosomes derived from human umbilical cord mesenchymal stem cells protect against cisplatin-induced ovarian granulosa cell stress and apoptosis in vitro. Sci Rep, 2017. 7(1): p. 2552.
40. Ling, B., et al., Effect of conditioned medium of mesenchymal stem cells on the in vitro maturation and subsequent development of mouse oocyte. Braz J Med Biol Res, 2008. 41(11): p. 978-85.
41. Sadighi-Moghaddam, B., et al., Mesenchymal Stem Cell Therapy Prevents Abortion in CBA/J×DBA/2 Mating. Reprod Sci, 2017: p. 1933719117737848.
42. Wang, L. L., et al., Effect of Human Umbilical Cord Mesenchymal Stem Cell Transplantation in a Rat Model of Preeclampsia. Reprod Sci, 2016. 23(8): p. 1058-70.
43. Qadri, M., et al., Role of CD44 in Regulating TLR2 Activation of Human Macrophages and Downstream Expression of Proinflammatory Cytokines. J Immunol, 2018. 200(2): p. 758-767.
44. Nedvetzki, S., et al., Reciprocal regulation of human natural killer cells and macrophages associated with distinct immune synapses. Blood, 2007. 109(9): p. 3776-85.
45. Scott, C. L., A. M. Aumeunier, and A. M. Mowat, Intestinal CD103+ dendritic cells: master regulators of tolerance? Trends Immunol, 2011. 32(9): p. 412-9.
46. Faas, M. M., F. Spaans, and P. De Vos, Monocytes and macrophages in pregnancy and pre-eclampsia. Front Immunol, 2014. 5: p. 298.
47. Hennessy, A., et al., A deficiency of placental IL-10 in preeclampsia. J Immunol, 1999. 163(6): p. 3491-5.
48. Makris, A., et al., Placental deficiency of interleukin-10 (IL-10) in preeclampsia and its relationship to an IL10 promoter polymorphism. Placenta, 2006. 27(4-5): p. 445-51.
49. McGowan, E. C., et al., Placental IL-10 dysregulation and association with bronchopulmonary dysplasia risk. Pediatr Res, 2009. 66(4): p. 455-60.
50. Szarka, A., et al., Circulating cytokines, chemokines and adhesion molecules in normal pregnancy and preeclampsia determined by multiplex suspension array. BMC Immunol, 2010. 11: p. 59.
51. Mutter, W. P. and S. A. Karumanchi, Molecular mechanisms of preeclampsia. Microvasc Res, 2008. 75(1): p. 1-8.
52. Wert, S. E., et al., Increased expression of thyroid transcription factor-1 (TTF-1) in respiratory epithelial cells inhibits alveolarization and causes pulmonary inflammation. Dev Biol, 2002. 242(2): p. 75-87.
53. Stahlman, M. T., M. E. Gray, and J. A. Whitsett, Expression of thyroid transcription factor-1 (TTF-1) in fetal and neonatal human lung. J Histochem Cytochem, 1996. 44(7): p. 673-8.
54. Willis, G. R., S. Kourembanas, and S. A. Mitsialis, Toward Exosome-Based Therapeutics: Isolation, Heterogeneity, and Fit-for-Purpose Potency. Front Cardiovasc Med, 2017. 4: p. 63.
55. Lee, C., et al., Exosomes mediate the cytoprotective action of mesenchym
56. Houser, B. L., et al., Two unique human decidual macrophage populations. J Immunol, 2011. 186(4): p. 2633-42.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating placental insufficiency in a female subject, the method comprising administering to the subject an effective amount of a mesenchymal stem cell (MSC) exosome.

2. The method of claim 1, wherein the isolated MSC exosome is isolated from MSC-conditioned media.

3. The method of claim 1 or claim 2, wherein the MSC is from Warton's Jelly or bone marrow.

4. The method of any one of claims 1-3, wherein the female subject is a human subject.

5. The method of any one of claims 1-4, wherein the female subject has preeclampsia.

6. The method of any one of claims 1-5, wherein the female subject has intrauterine inflammation.

7. The method of any one of claims 1-6, wherein the female subject has infertility.

8. The method of any one of claims 1-7, wherein the placental insufficiency results in fetal growth restriction and/or fetal loss.

9. The method of any one of claims 1-8, wherein the MSC exosome is administered once.

10. The method of any one of claims 1-8, wherein the MSC exosome is administered repeatedly.

11. The method of any one of claims 1-10, wherein the MSC exosome is administered via intravenous injection.

12. The method of any one of claims 1-10, wherein the MSC exosome is administered via intrauterine injection.

13. The method of any one of claims 1-12, wherein the MSC exosome is administered antepartum.

14. The method of any one of claims 1-12, wherein the MSC exosome is administered intrapartum.

15. The method of any one of claims 1-14, wherein the MSC exosome reduces intrauterine inflammation.

16. The method of any one of claims 1-15, wherein the MSC exosome reverses placental insufficiency.

17. The method of any one of claims 1-16, wherein the MSC exosome reduces the likelihood of fetal growth restriction and/or fetal loss.

18. Use of a mesenchymal stem cell (MSC) exosome to treat placental insufficiency in a female subject.

19. A method of treating fetal growth restriction, the method comprising administering to a fetus in a pregnant female subject an effective amount of a mesenchymal stem cell (MSC) exosome.

20. The method of claim 19, wherein the isolated MSC exosome is isolated from MSC-conditioned media.

21. The method of claim 19 or claim 20, wherein the MSC is from Warton's Jelly or bone marrow.

22. The method of any one of claims 19-21, wherein the fetus is a human fetus.

23. The method of any one of claims 19-22, wherein the fetal growth restriction is caused by placental insufficiency of the pregnant female subject.

24. The method of any one of claims 19-23, wherein the MSC exosome is administered via intravenous injection to the pregnant female subject.

25. The method of any one of claims 19-23, wherein the MSC exosome is administered to the amniotic fluid of the pregnant female subject.

26. The method of any one of claims 19-23, wherein the MSC exosome is administered via injection into the umbilical vein of the umbilical cord.

27. The method of any one of claims 19-25, wherein the MSC exosome is administered once.

28. The method of any one of claims 19-25, wherein the MSC exosome is administered repeatedly.

29. The method of any one of claims 19-28, wherein the MSC exosome is administered antenatal.

30. The method of any one of claims 19-28, wherein the MSC exosome is administered intrapartum.

31. The method of any one of claims 19-28, wherein the MSC exosome is administered perinatal.

32. The method of any one of claims 19-31, wherein the MSC exosome reduces the likelihood of fetal loss.

33. The method of any one of claims 19-31, wherein the MSC exosome ameliorates pre-eclampsia-related alterations in fetal lung development.

34. Use of a mesenchymal stem cell (MSC) exosome to treat fetal growth restriction of a fetus in a pregnant female subject.

35. A method of treating infertility, the method comprising administering to a female subject in need thereof an effective amount of a mesenchymal stem cell (MSC) exosome.

36. The method of claim 35, wherein the isolated MSC exosome is isolated from MSC-conditioned media.

37. The method of claim 35 or claim 36, wherein the MSC is from Warton's Jelly or bone marrow.

38. The method of any one of claims 35-37, wherein the subject is a human subject.

39. The method of any one of claims 35-38, wherein the female subject has history of pelvic inflammatory disease, advanced maternal age, obesity, metabolic or cardiovascular disease, history of endometriosis or fibroids, chronic maternal hypertension, polycystic ovary syndrome, and/or history of sexually transmitted infections with secondary scarring.

40. The method of any one of claims 35-39, wherein the subject has intrauterine inflammation.

41. The method of any one of claims 35-40, wherein the subject has placental insufficiency.

42. The method of any one of claims 35-41, wherein the MSC exosome is administered once.

43. The method of any one of claims 35-41, wherein the MSC exosome is administered repeatedly.

44. The method of any one of claims 35-43, wherein the MSC exosome is administered via intravenous injection.

45. The method of any one of claims 35-43, wherein the MSC exosome is administered via intrauterine injection.

46. Use of a mesenchymal stem cell (MSC) exosome to treat infertility in a female subject.
